(19) United States
(12) Patent Application Publication
(10) Pub. No.: US 2025/0180536 A1
(43) Pub. Date: Jun. 5, 2025

(54) Water quality management device, operation method thereof, and water quality management method

(71) Applicant: SK Planet Co., Ltd., Seongnam-si (KR)

(72) Inventor: Yeonghyeon PARK, Suwon-si (KR)

(21) Appl. No.: 19/047,636

(22) Filed: Feb. 7, 2025

Related U.S. Application Data
(63) Continuation of application No. PCT/KR2023/019415, filed on Nov. 29, 2023.

(30) Foreign Application Priority Data
Mar. 6, 2023 (KR) 10-2023-0028904
Mar. 6, 2023 (KR) 10-2023-0028905
Mar. 6, 2023 (KR) 10-2023-0028906

(51) Int. Cl. G01N 33/18; G06V 10/762; G06V 10/764; G06V 20/70

(52) U.S. Cl. CPC G01N 33/18; G06V 10/762; G06V 10/764; G06V 20/70

(57) ABSTRACT

The present invention may provide a water quality management device including a spectral camera, a memory, and a processor, and an operating method thereof. The processor is configured to collect the plurality of spectral images for at least a portion of the stored water in response to a predefined event occurrence, classify the plurality of spectral images into a suspended material data set and a non-suspended material data set based on a presence or absence of a suspended material in the spectral images, perform learning to generate a water quality detection model used to detect water quality by using at least a portion of the suspended material data set and the non-suspended material data set, and store the water quality detection model generated through the learning.

### CROSS-REFERENCE TO RELATED APPLICATIONS

This is a bypass continuation of International PCT Application No. PCT/KR2023/019415, filed on Nov. 29, 2023, which claims priority to Korean Patent Application No. 10-2023-0028904, filed on Mar. 6, 2023, Korean Patent Application No. 10-2023-0028905, filed on Mar. 6, 2023, and Korean Patent Application No. 10-2023-0028906 filed on Mar. 6, 2023, which are incorporated by reference herein in their entirety.

### TECHNICAL FIELD

The present invention relates to water quality management, and more particularly, to a water quality management technology that performs water quality management using spectral data.

### BACKGROUND ART

In order for urban facilities and industrial facilities to operate normally, the supply and treatment of water are essential. In this regard, water facilities are provided and configured to store, purify, supply, and post-treat water. If the quality management of water stored in water facilities is not properly performed, there is a risk that various diseases will spread rapidly.

Accordingly, in order to manage water quality accurately, a scheme of checking the current water quality status accurately is required. In response to this demand, various interests are focused on the quality management of water in water facilities for the operation of urban facilities and industrial facilities. In relation to water quality management, various sensors capable of sensing the water quality of stored water are installed typically, and the water quality is checked by analyzing sensing information collected by the sensors.

However, since the sensors of this sensor operation scheme are disposed only in specific locations to sense the water quality of stored water, the typical water quality management method has a problem in that only very local water quality is checked. In addition, since this sensor operation scheme only performs a rough water quality check of the stored water, a more accurate water quality management control method is required.

### SUMMARY

The present invention is intended to provide a water quality management device that can be used to more accurately determine the quality of water stored in a tank, a method of operating the same, and a water quality management method.

However, the objects of the present invention are not limited to the above objects, and other objects not mentioned can be clearly understood from the description below.

To achieve the above objects, a water quality management device may include a spectral camera acquiring a plurality of spectral image related to stored water, a memory, and a processor functionally connected to the spectral camera and the memory. The processor may be configured to collect the plurality of spectral images for at least a portion of the stored water in response to a predefined event occurrence, classify the plurality of spectral images into a suspended material data set and a non-suspended material data set based on a presence or absence of a suspended material in the spectral images, perform learning to generate a water quality detection model used to detect water quality by using at least a portion of the suspended material data set and the non-suspended material data set, and store the water quality detection model generated through the learning.

Specifically, the processor may be configured to assign indexes to the plurality of spectral images acquired through the spectral camera, and perform labeling on the plurality of spectral images for the assigned indexes.

Specifically, the processor may be configured to check whether a label exists for a spectral image of a specific index among the assigned indexes, and if there is no label, perform an integrity check of the spectral image of the specific index, check a presence or absence of a suspended material in the spectral image that has undergone the integrity check, and perform labeling for the presence or absence of the suspended material.

Specifically, the processor may be configured to perform the integrity check based on at least one of an RGB image, dissolved oxygen, and mixed liquor suspended solids (MLSS) collected together at a time when the spectral image of the specific index is collected in relation to the integrity check.

Specifically, the processor may be configured to, in relation to the suspended material check, output at least one of an RGB image, dissolved oxygen, and mixed liquor suspended solids (MLSS) collected together at a time when the spectral image of the specific index among the assigned indexes is collected, and confirm the presence or absence of the suspended material in response to a user input.

Specifically, the processor may be configured to divide the suspended material data set into a training data set and a validation data set with a predefined ratio, generate the water quality detection model based on the training data set, and perform a performance evaluation on the water quality detection model by using the validation data set.

A water quality management method according to the present invention includes, by a processor of a water quality management device, collecting a plurality of spectral images for at least a portion of stored water in response to a predefined event occurrence; classifying the plurality of spectral images into a suspended material data set and a non-suspended material data set based on a presence or absence of a suspended material in the spectral images; performing learning to generate a water quality detection model used to detect water quality by using at least a portion of the suspended material data set and the non-suspended material data set; and storing the water quality detection model generated through the learning.

Specifically, classifying may include assigning indexes to the plurality of spectral images acquired through the spectral camera, and performing labeling on the plurality of spectral images for the assigned indexes, labeling may include checking whether a label exists for a spectral image of a specific index among the assigned indexes, and if there is no label, performing an integrity check of the spectral image of the specific index, checking a presence or absence of a suspended material in the spectral image that has undergone the integrity check, and performing labeling for the presence or absence of the suspended material, and checking the presence or absence of the suspended material may include outputting at least one of an RGB image, dissolved oxygen, and mixed liquor suspended solids (MLSS) collected together at a time when the spectral image of the specific index is collected, and confirming the presence or absence of the suspended material in response to a user input.

Specifically, classifying may include dividing the suspended material data set into a training data set and a validation data set with a predefined ratio, generating the water quality detection model based on the training data set, and performing a performance evaluation on the water quality detection model by using the validation data set.

A water quality management device according to the present invention may include a spectral camera acquiring a plurality of spectral image related to stored water, a memory storing a water quality detection model generated based on the plurality of spectral images, and a processor functionally connected to the spectral camera and the memory. For example, the processor may be configured to collect a spectral image for water quality management of the stored water at a current time, calculate a non-suspended material score for the spectral image at the current time by using the water quality detection model, perform classification for water quality management for the spectral image if the calculated non-suspended material score exceeds a predefined threshold value, and control the water quality management based on a value of the classification.

Specifically, the memory may include a spectral library including at least one of a suspended material data archive and a non-suspended material data archive, and the processor may be configured to calculate the non-suspended material score based on at least one of the suspended material data archive or the non-suspended material data archive for the spectral image.

Specifically, the memory may include a spectral library including a suspended material data archive, and the processor may be configured to perform clustering on the spectral image at the current time, and calculate the non-suspended material score by comparing the cluster resulting from the clustering with clusters pre-stored in the suspended material data archive of the memory.

Specifically, the processor may be configured to store information about the spectral image in a non-suspended material data archive of the memory if the non-suspended material score is less than the predefined threshold value.

Specifically, the processor may be configured to, in relation to performing the classification, define a purification intensity differently depending on a degree of a suspended material included in the spectral image, and produce a classification value of the current spectral image based on classification values pre-stored in the memory.

Specifically, the processor may be configured to transmit a warning message including the purification intensity corresponding to the classification value to a water purification device capable of performing water purification on the stored water or to an administrator terminal of an administrator managing the water purification device.

A water quality management method according to the present invention may include, by a processor of a water quality management device, collecting a spectral image related to stored water at a current time; calculating a non-suspended material score for the spectral image at the current time by using a water quality detection model generated based on a plurality of spectral images; performing classification for water quality management for the spectral image if the calculated non-suspended material score exceeds a predefined threshold value; and controlling the water quality management based on a value of the classification.

Specifically, calculating the non-suspended material score may include, based on a spectral library including at least one of a suspended material data archive and a non-suspended material data archive, calculating the non-suspended material score based on at least one of the suspended material data archive or the non-suspended material data archive for the spectral image.

Performing the classification may include defining a purification intensity differently depending on a degree of a suspended material included in the spectral image, and producing a classification value of the current spectral image based on classification values pre-stored in the memory.

Controlling may include transmitting a warning message including the purification intensity corresponding to the classification value to a water purification device capable of performing water purification on the stored water or to an administrator terminal of an administrator managing the water purification device.

A water quality management device according to the present invention may include a spectral camera acquiring a plurality of spectral image related to stored water, a memory storing a spectral library that classifies data on a suspended material relevance of the plurality of spectral images and storing image classification values derived through deep learning for the plurality of spectral images, and a processor functionally connected to the spectral camera and the memory. The processor may be configured to collect a current spectral image for water quality management of the stored water at a current time, estimate the water quality of the current spectral image by using at least one of a process of identifying the suspended material relevance of the current spectral image by using the spectral library and a process of identifying an image classification value of the current spectral image by using the image classification values, and based on a result of estimating the water quality, determine whether to perform purification processing for the stored water at the current time.

Specifically, the processor may be configured to measure a non-suspended material score for the current spectral image by using the spectral library, if the non-suspended material score exceeds a threshold value, generate control information for the purification processing for the stored water at the current time, and transmit the control information to a purification device for the purification processing.

Specifically, the processor may be configured to measure a non-suspended material score for the current spectral image by using the spectral library, if the non-suspended material score exceeds a threshold value, output a screen for checking whether to end a process related to the water quality management, and if there is no user input related to the end of the process, store the current spectral image in a non-suspended material data archive.

Specifically, the processor may be configured to measure a non-suspended material score for the current spectral image by using the spectral library, and if the non-suspended material score exceeds a threshold value, store the current spectral image in a non-suspended material data archive.

Specifically, the processor may be configured to compare the image classification values with the image classification value for the current spectral image, if the image classification value for the current spectral image has a value requiring purification processing as a result of comparison, generate control information for the purification processing, and transmit the control information to a purification device for the purification processing.

A water quality management method according to the present invention may include, by a processor of a water quality management device, loading, from a memory, a spectral library that classifies data on a suspended material relevance of a plurality of spectral images, and image classification values derived through deep learning for the plurality of spectral images; by the processor, collecting a current spectral image for water quality management of stored water at a current time by controlling a spectral camera; estimating the water quality of the current spectral image by using at least one of a process of water quality estimation based on the suspended material relevance of the current spectral image using the spectral library and a process of water quality estimation based on an image classification value of the current spectral image calculated based on the image classification values; and based on a result of estimating the water quality, determine whether to perform purification processing for the stored water at the current time.

Specifically, estimating may include measuring a non-suspended material score for the current spectral image by using the spectral library, and if the non-suspended material score exceeds a threshold value, determining the water quality of the stored water at the current time as requiring purification processing, or comparing the image classification values with the image classification value for the current spectral image, and if the image classification value for the current spectral image has a value requiring purification processing as a result of comparison, determining the water quality of the stored water at the current time as requiring purification processing.

Specifically, the method may further include, if the water quality is determined as requiring purification processing, creating control information for the purification processing, and transmitting the created control information to a purification device for the purification processing.

According to the present invention, it is possible to more accurately process real-time water quality management by using spectral data, and provide a water quality detection model necessary for more efficient water quality management.

Compared to the typical sensor operation scheme, the present invention can quickly and accurately determine a wider range of water quality as needed.

The present invention can support appropriately performing additional measures necessary for water quality management at the current time.

In addition, the present invention can perform water quality observation more accurately, and support more rapid water quality management control by allowing real-time check at a time desired by an administrator.

In addition, various effects other than the effects described above can be directly or implicitly disclosed in the detailed description according to embodiments of the present invention to be described later.

### BRIEF DESCRIPTION OF DRAWINGS

FIG. 1 is a diagram showing an example of a water quality management system according to the first embodiment of the present invention.

FIG. 2A is a diagram showing an example of components of a water quality management device according to the first embodiment of the present invention.

FIG. 2B is a diagram showing an example of a spectral data average according to the amount of each suspended material.

FIG. 2C is a diagram showing an example of a spectral data standard deviation according to the amount of each suspended material.

FIG. 3 is a diagram showing an example of components of a main server device according to the first embodiment of the present invention.

FIG. 4 is a diagram showing an example of a labeling method for training a water quality detection model according to the first embodiment of the present invention.

FIG. 5 is a diagram showing an example of a learning preparation method for a water quality detection model according to the first embodiment of the present invention.

FIG. 6 is a diagram showing another example of a learning preparation method for a water quality detection model according to the first embodiment of the present invention.

FIG. 7 is a diagram showing an example of a learning method for a water quality detection model according to the first embodiment of the present invention.

FIG. 8 is a diagram showing an example of a water quality management system according to the second embodiment of the present invention.

FIG. 9 is a diagram showing an example of components of a water quality management device according to the second embodiment of the present invention.

FIG. 10 is a diagram showing an example of components of a main server device according to the second embodiment of the present invention.

FIG. 11 is a diagram showing an example of a suspended material detection model operation according to the second embodiment of the present invention.

FIG. 12 is a diagram showing an example of a spectral library preparation method according to the second embodiment of the present invention.

FIG. 13 is a diagram showing another example of a spectral library preparation method according to the second embodiment of the present invention.

FIG. 14 is a diagram showing an example of a water quality management method according to the second embodiment of the present invention.

FIG. 15 is a diagram showing an example of a water quality management system according to the third embodiment of the present invention.

FIG. 16 is a diagram showing an example of components of a water quality management device according to the third embodiment of the present invention.

FIG. 17 is a diagram showing an example of components of a main server device according to the third embodiment of the present invention.

FIG. 18 is a diagram showing an example of a spectral library preparation method according to the third embodiment of the present invention.

FIG. 19 is a diagram showing another example of a spectral library preparation method according to the third embodiment of the present invention.

FIG. 20 is a diagram showing an example of a water quality management method according to the third embodiment of the present invention.

### DETAILED DESCRIPTION OF EMBODIMENTS

Now, embodiments of the present invention will be described in detail with reference to the accompanying drawings.

However, in the following description and the accompanying drawings, well known functions and components may not be described nor illustrated in detail to avoid obscuring the subject matter of the present invention. In addition, identical components are indicated with the same reference numerals as much as possible throughout the drawings

The terms or words used in the following description and drawings should not be interpreted as limited to their usual or dictionary meanings and should be interpreted as meanings and concepts that conform to the technical idea of the present invention based on the principle that the inventor can appropriately define the concept of the terms to best describe his or her invention. Therefore, embodiments described herein are only the most preferred embodiments of the present invention and do not represent all of the technical ideas of the present invention. Thus, it should be understood that there may be various equivalents and modified examples that can replace the embodiments at the time of filing this application.

In addition, terms including ordinal numbers such as first, second, etc. are used to describe various elements only for the purpose of distinguishing one element from another, and are not used to limit such elements. For example, without departing from the scope of the present invention, a second element may be named a first element, and similarly, a first element may also be named a second element.

In addition, terms used herein are only for describing specific embodiments and do not limit the present invention. The singular expression includes the plural expression unless the context clearly indicates otherwise. Also, the terms such as “comprise” and “include” used herein are intended to specify the presence of features, numerals, steps, operations, elements, components, or combinations thereof, which are disclosed herein, and should not be construed to exclude in advance the possibility of the presence or addition of other features, numerals, steps, operations, elements, components, or combinations thereof.

In addition, the terms such as “unit” and “module” used herein refer to a unit that processes at least one function or operation and may be implemented with hardware, software, or a combination of hardware and software. In addition, the terms “a”, “an”, “one”, “the”, and similar terms may be used as both singular and plural meanings in the context of describing the present invention (especially in the context of the following claims) unless the context clearly indicates otherwise.

In addition to the terms mentioned above, specific terms used in the following description are provided to help understanding of the present invention, and the use of such specific terms may be changed to other forms without departing from the technical meaning of the present invention.

Also, embodiments within the scope of the present invention include computer-readable media having computer-executable instructions or data structures stored on computer-readable media. Such computer-readable media can be any available media that is accessible by a general purpose or special purpose computer system. By way of example, such computer-readable media may include, but not limited to, RAM, ROM, EPROM, CD-ROM or other optical disk storage, magnetic disk storage or other magnetic storage devices, or any other physical storage medium that can be used to store or deliver certain program codes formed of computer-executable instructions, computer-readable instructions or data structures and which can be accessed by a general purpose or special purpose computer system.

Hereinafter, a water quality management system that supports the generation of a model related to water quality management of the present invention and the types and roles of respective components included therein will be described.

### First Embodiment

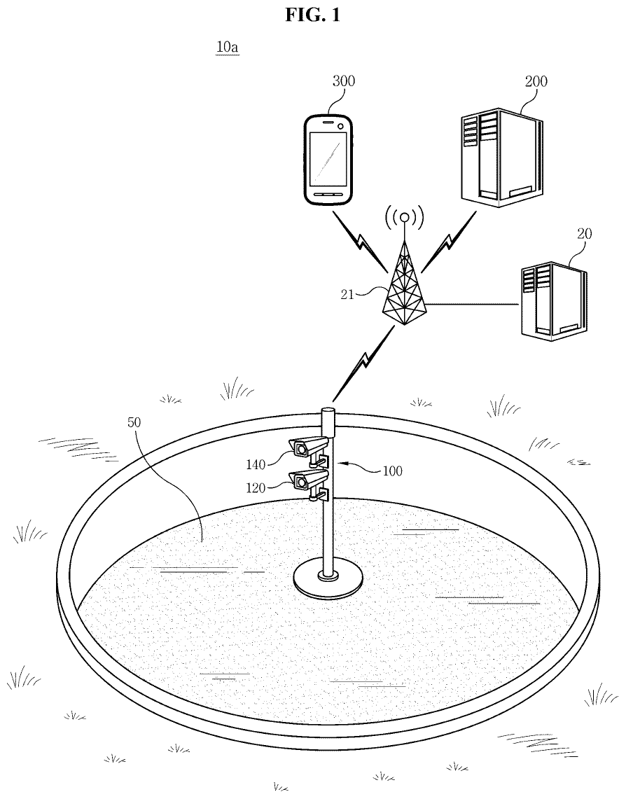

FIG. 1 is a diagram showing an example of a water quality management system according to the first embodiment of the present invention.

Referring to FIG. 1, the water quality management system 10a of the present invention may include stored water 50 (e.g., water stored in a tank), a water quality management device 100, a base station 21, a relay server device 20, a main server device 200, and an administrator terminal 300. Here, the structure in which the water quality management system 10a includes the base station 21 for communication between the water quality management device 100 and the relay server device 20, the main server device 200, or the administrator terminal 300 is exemplified, but the present invention is not limited thereto. For example, without the base station 21, the water quality management device 100 and the main server device 200 (or the relay server device 20) may also be directly connected via a wired cable or a short-range wireless communication scheme. In this case, the base station 21 may be omitted from the water quality management system 10a. In addition, although it is exemplified that the relay server device 20 is included in the water quality management system 10a, the present invention is not limited thereto. For example, the relay server device 20 may be omitted or provided as being integrated with the main server device 200.

The stored water 50 is water stored in a tank prepared to have a certain size and capable of storing water, and the water tank may be at least one of various tank types that can store water, such as a sewage tank of a sewage treatment plant or a water supply tank for water supply treatment. Therefore, the stored water 50 may be water stored in a sewage tank, water stored in a water supply tank, or the like. The stored water 50 in the present invention may be classified into water including a suspended material of a certain size or larger or water including no suspended material, depending on conditions, for example. The stored water 50 including a suspended material may be purified through a purification process. The stored water 50 may be located within a shooting angle at which a spectral camera 120 and an RGB camera 140 disposed in the water quality management device 100 can photograph.

The water quality management device 100 may include the spectral camera 120 that collects spectral images of the stored water 50, the RGB camera 140 that collects RGB images of the stored water 50, and a mounting structure that mounts the spectral camera 120 and the RGB camera 140. Although it is described that the water quality management device 100 includes both the spectral camera 120 and the RGB camera 140, the present invention is not limited thereto. For example, the water quality management device 100 may include only the spectral camera 120. Additionally, the water quality management device 100 according to the present invention may further include at least one sensor that can collect sensing information related to the water quality of the stored water 50 in addition to the spectral images. According to the occurrence of a predefined event or a designated cycle or time point, the water quality management device 100 may generate a water quality detection model used for water quality detection of the stored water 50 based on at least some of the spectral image collected by the spectral camera 120, the RGB image collected by the RGB camera 140, and the sensing information collected by the at least one sensor. In this regard, the water quality management device 100 trains a model related to water quality measurement based on collected data (e.g., at least one of the spectral image collected by the spectral camera 120, the RGB image collected by the RGB camera 140, and the sensing information collected by the at least one sensor).

Meanwhile, the water quality management device 100 may be configured to transmit the collected data (e.g., at least one of the spectral image collected by the spectral camera 120, the RGB image collected by the RGB camera 140, and the sensing information collected by the at least one sensor) related to the stored water 50 to the main server device 200. In this case, the water quality management device 100 may form a communication channel with the main server device 200 through the base station 21. The water quality management device 100 may transmit the collected data (e.g., at least one of the spectral image collected by the spectral camera 120, the RGB image collected by the RGB camera 140, and the sensing information collected by the at least one sensor) to the main server device 200.

The base station 21 may be spaced apart from the water quality management device 100 by a certain distance and disposed geographically at a distance or location where a wireless communication channel can be formed with the water quality management device 100. Alternatively, the base station 21 may be connected to the water quality management device 100 by wire. Additionally, the base station 21 may support forming at least one of a communication channel between the water quality management device 100 and the main server device 200, a communication channel between the water quality management device 100 and the relay server device 20, a communication channel between the relay server device 20 and the main server device 200, a communication channel between the water quality management device 100 and the administrator terminal 300, and a communication channel between the main server device 200 and the administrator terminal 300.

The main server device 200 may receive information collected by the water quality management device 100 and generate a water quality detection model based on the received information. In this regard, the main server device 200 may form a communication channel with the water quality management device 100 through the base station 21 (or directly without the base station 21). Meanwhile, if the water quality management device 100 is configured to independently generate the water quality detection model, the main server device 200 may be omitted. The main server device 200 may receive collected data (e.g., at least one of a spectral image, an RGB image collected together at the spectral image collection time, and sensing information) from the water quality management device 100 at the time of occurrence of a predefined event. The main server device 200 may enable the water quality detection model to learn based on the received collected data (e.g., at least one of the spectral image collected by the spectral camera 120, the RGB image collected by the RGB camera 140, and sensing information collected by the at least one sensor). Thereafter, the main server device 200 may provide the learning-completed water quality detection model to the water quality management device 100. The main server device 200 may transmit at least a part of the process related to learning (or generation) of the water quality detection model to a designated administrator terminal 300 (or user terminal) or output it through a connected display device.

Although it is described above that at least one sensor is included in the water quality management device 100, the present invention is not limited thereto. For example, the at least one sensor may be placed at a specific location of the stored water 50 without being included in the water quality management device 100, have a communication module on its own, and be connected to the relay server device 20 via the base station 21. Here, the communication module included in the at least one sensor may be connected to the relay server device 20 via a wired cable, or may be formed as a wireless communication module and connected to the relay server device 20 via the base station 21. The at least one sensor may collect sensing information on the stored water 50 according to predefined schedule information, and transmit the collected sensing information to the relay server device 20.

The relay server device 20 may be connected to at least one sensor, and transmit sensing information collected from the at least one sensor to at least one of the water quality management device 100 and the main server device 200 at regular intervals or in real time. Alternatively, the relay server device 20 may receive control information regarding at least one sensor from at least one of the water quality management device 100 and the main server device 200, and control the at least one sensor based on the received control information to collect sensing information. Meanwhile, as mentioned above, the relay server device 20 may be integrated with the main server device 200 or, if at least one sensor is included in the water quality management device 100, may be omitted.

The administrator terminal 300 may form a communication channel with at least one of the water quality management device 100, the relay server device 20, and the main server device 200 through the base station 21. The administrator terminal 300 may request the water quality management device 100 to generate the water quality detection model. Alternatively, the administrator terminal 300 may request the main server device 200 to generate the water quality detection model.

As described above, the water quality management system 10a according to the first embodiment of the present invention can acquire a plurality of spectral images of the stored water 50 at the time of predefined event occurrence, generate a water quality detection model based on learning about the acquired spectral images, and provide the generated model. In particular, the water quality management system 10a of the present invention can collect information about the presence or not of a suspended material that has a significant impact on water quality, the shape and extent of the suspended material, and construct a water quality detection model related to the suspended material.

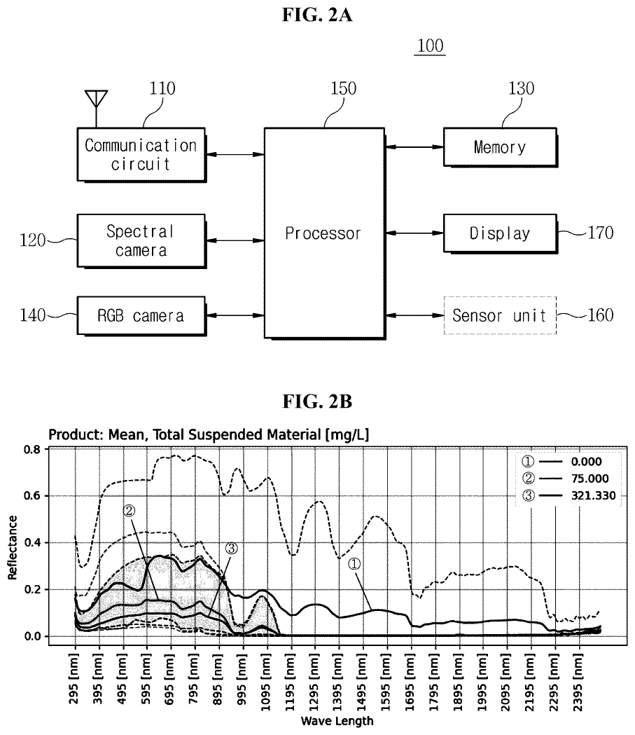

FIG. 2A is a diagram showing an example of components of a water quality management device according to the first embodiment of the present invention. FIG. 2B is a diagram showing an example of a spectral data average according to the amount of each suspended material. FIG. 2C is a diagram showing an example of a spectral data standard deviation according to the amount of each suspended material.

In the following description, a form in which the relay server device 20 is integrated with the main server device 200 is exemplified. However, the present invention is not limited thereto, and the relay server device 20 may be separately configured in the water quality management system 10a for the operation of a sensor unit 160 including at least one sensor.

Referring to FIG. 2A, the water quality management device 100 according to the first embodiment of the present invention may include a communication circuit 110, a spectral camera 120, a memory 130, an RGB camera 140, a sensor unit 160, a display 170, and a processor 150. Here, if the water quality management device 100 is configured to generate a water quality detection model by using only spectral images, at least one of the RGB camera 140 and the sensor unit 160 may be omitted. In addition, the water quality management device 100 may further include a mounting structure for mounting the spectral camera 120 so that the spectral camera 120 can be oriented to and capture at least a portion of the stored water 50. In addition, the water quality management device 100 may further include a power supply (e.g., a permanent power supply or a battery) required for the operation of at least one of the above-described components, for example, the communication circuit 110, the spectral camera 120, the memory 130, the RGB camera 140, the sensor unit 160, the display 170, and the processor 150. Meanwhile, at least one of the components of the water quality management device 100 may be omitted or integrated into another component.

The communication circuit 110 may form a communication channel with the main server device 200 via the base station 21. Alternatively, the communication circuit 110 may form a communication channel directly with the main server device 200 without going through the base station 21. If the main server device 200 is designed to generate the water quality detection model for water quality management according to the first embodiment of the present invention, the communication circuit 110 may transmit spectral images collected by the spectral camera 120 to the main server device 200. On the other hand, the generation of the water quality detection model may be performed independently by the water quality management device 100. The communication circuit 110 may receive an artificial neural network algorithm necessary for generating the water quality detection model from the main server device 200.

The spectral camera 120 may be disposed to capture a spectral image for at least a portion (or at least a point) of the stored water 50. The spectral camera 120 may capture a plurality of spectral images of the stored water 50 at a control time of the processor 150. For example, when a predefined scheduled event occurs, the spectral camera 120 may capture a plurality of spectral images related to the stored water 50 in response to the occurrence of the event. For example, under the control of the processor 150 (or in response to a request of the main server device 200), the spectral camera 120 may acquire at least one spectral image related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). Alternatively, the spectral camera 120 may capture spectral images related to the stored water 50 according to a predefined regular cycle. The spectral images collected by the spectral camera 120 may be temporarily or semi-permanently stored in the memory 130.

The memory 130 may store at least one program or data required for the operation of the water quality management device 100. For example, the memory 130 may store a control program required for operating the spectral camera 120, a plurality of spectral images acquired by the spectral camera 120, and a water quality detection model generated based on the plurality of spectral images. Additionally, the memory 130 may store at least one RGB image and at least one sensing information used for generating the water quality detection model. In addition, the memory 130 may store an artificial neural network algorithm or a machine learning algorithm required for the water quality detection model.

The RGB camera 140 may be disposed to have the same shooting angle or face the same shooting point (or portion) as the shooting angle or shooting point of the spectral camera 120. The RGB camera 140 may acquire an RGB image of the stored water 50 while being synchronized with the time at which the spectral camera 120 captures a spectral image. Therefore, the RGB camera 140 may acquire a plurality of RGB images related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). The acquired plurality of RGB images may be temporarily stored in the memory 130 and, in response to the control of the processor 150, may be used to generate a water quality detection model, or transmitted to a designated device. For example, the acquired plurality of RGB images may be output through the display 170 of the water quality management device 100.

The sensor unit 160 may be disposed in the water quality management device 100 and collect at least one type of sensor information related to the water quality of the stored water 50 at the time when the spectral camera 120 acquires the spectral image. For example, the sensor unit 160 may include at least one of a dissolved oxygen (DO) sensor, a mixed liquor suspended solids (MLSS) sensor, a biochemical oxygen demand (BOD) sensor, and a chemical oxygen demand (COD) sensor, which can directly measure water quality information. In addition, the sensing information collected by the sensor unit 160 may be temporarily stored in the memory 130 and, depending on the design form, may be used in a water quality detection model or transmitted to the main server device 200. Meanwhile, at least one sensor included in the sensor unit 160 may be connected to the relay server device 20 and configured to collect sensing information in response to the control of the relay server device 20 and then transmit the sensing information to at least one of the water quality management device 100 or the main server device 200. Alternatively, at least part of the sensing information may be output through the display 170.

The display 170 may output various information screens required for the operation of the water quality management device 100. For example, the display 170 may output a screen related to the operation of at least one of the spectral camera 120 and the RGB camera 140 of the water quality management device 100. The display 170 may output at least in part the operating information of the sensor unit 160 and the sensing information collected by the sensor unit 160. In addition, the display 170 may output a screen related to learning of a water quality detection model (or generative model). The display 170 may include a touch screen that supports a user input function.

The processor 150 may control the spectral camera 120 to acquire a plurality of spectral images related to the stored water 50 in response to a predefined event or a request from the main server device 200. The processor 150 may perform the generation of a water quality detection model based on the acquired spectral images.

For example, the processor 150 may generate a water quality detection model based on a suspended material. The processor 150 may collect a plurality of spectral images of the stored water 50 by using the spectral camera 120 and may generate a model capable of determining the presence or absence of a suspended material (SM) by analyzing the collected plurality of spectral images. For example, referring to FIGS. 2B and 2C, in the process of analyzing the acquired spectral image, the processor 150 may determine that a suspended material exists in the spectral image if the wavelength is relatively short compared to predefined criteria and the fluctuation range of reflectance is less than the predefined criteria when observing the situation. The processor 150 may determine that a suspended material does not exist in the spectral image if the wavelength is relatively long compared to the predefined criteria, the overall reflectance is high, and the variation range is greater than the predefined criteria. The predefined criteria may be the average value of filtering (or clustering) for the acquired spectral images or may be defined by the setting value of the administrator involved in model generation. For example, as shown in FIGS. 2B and 2C, the consistency of the spectral images in the case where a suspended material exists may be higher than the consistency of the spectral images in the case where a suspended material does not exist. Thus, the processor 150 may use the high-consistent spectral images with suspended materials for learning of a generative model corresponding to the water quality detection model.

In relation to the generative model learning, the processor 150 may use the Auto-Encoder or the Generative Adversarial Network among artificial neural networks for deep learning. The processor 150 may use a traditional machine learning model in addition to a deep learning algorithm, and the present invention is not limited to deep learning and machine learning models. The processor 150 may output a user UI (e.g., a screen displaying a list of training data, a screen for selecting at least one item included in the list) through the display 170 so that a user (or an administrator involved in generative model learning) can manually designate a list of training data. The processor 150 may process label designation for each data sample in response to a user input. In the labeling operation, the processor 150 may include at least spectral data (or spectral image) in the label, and may also output at least one type of sensing information collected by the sensor unit 160 to the display 170 so that the user can refer to it during the labeling process.

In another example, the processor 150 may support semi-automatic designation of a label by a criterion allowed by the user (or the administrator). In applying the criterion, the processor 150 may include a spectral image (or spectral data) in the corresponding labeled data and output at least part of the sensing information collected by the sensor unit 160 to the display 170 for reference. In relation to the semi-automatic designation, the processor 150 may provide a specific criterion value (e.g., at least some of values obtained by multiplying the average value and the standard deviation of frequency values of spectral images by a predefined coefficient value) for determining the presence or not of a suspended material, or may output a screen on the display 170 that allows the user to designate the specific criterion value, and set the specific criterion value for determining the presence or not of a suspended material in the spectral image by a user input. When the criterion value is determined, the processor 150 may analyze the spectrum of spectral images to check whether frequency distribution is less than or greater than the criterion value, and thereby automatically determine the presence or not of a suspended material.

In relation to a semi-automatic spectral library construction through a suspended material discrimination model, if the processor 150 determines that a suspended material exists in the spectral image according to the suspended material discrimination method described above, the processor 150 may store the spectral image determined to have a suspended material in a suspended material data archive area (or a separate suspended material archive) of the memory 130. If the processor 150 determines that a suspended material does not exist, the processor 150 may store the spectral image in the non-suspended material (non-SM) data archive area (or non-suspended material data archive) of the memory 130. If the number of data held by the suspended material archive exceeds a predefined certain amount or a user request occurs, the processor 150 may cluster the data stored in the archive by similar characteristics. An example of a clustering method may be K-means clustering, but the present invention is not limited thereto. Based on settings or user input, the processor 150 may perform the same clustering process on data stored in the non-suspended material (non-SM) data archive. When clustering is completed, the processor 150 may output the clustering result to the display 170 to allow the user (or administrator) to check the clustering result, and may confirm the information indicated by the cluster in response to a user input. In this process, the information output to the display 170 may include the result of a specific cluster, a spectral image corresponding to the cluster, and/or sensing information collected together when the spectral image is collected. In addition, the processor 150 may output a reference cluster in which a suspended material exists to the display 170. When the user confirms the cluster information, the processor 150 may add the confirmed cluster to the spectral library. For example, the confirmed information may include one or more of information necessary for water quality management, such as water quality status, types of suspended materials, and measures to be taken for water quality management. Based on setting or a user request, the processor 150 may support determining information by referring to other data (e.g., at least some data of RGB images and sensing information for the stored water 50) existing in a hyperspectral library. In this regard, the processor 150 may also output a reference RGB image in which a suspended material exists or reference sensing information in which a suspended material exists to the display 170. For example, based on setting or a user input, the processor 150 may delete or overwrite information existing in the hyperspectral library. Based on setting or a user request, the processor 150 may support clustering of at least one of the suspended material data archive and the non-suspended material data archive and generation of a clustering-based water quality detection model.

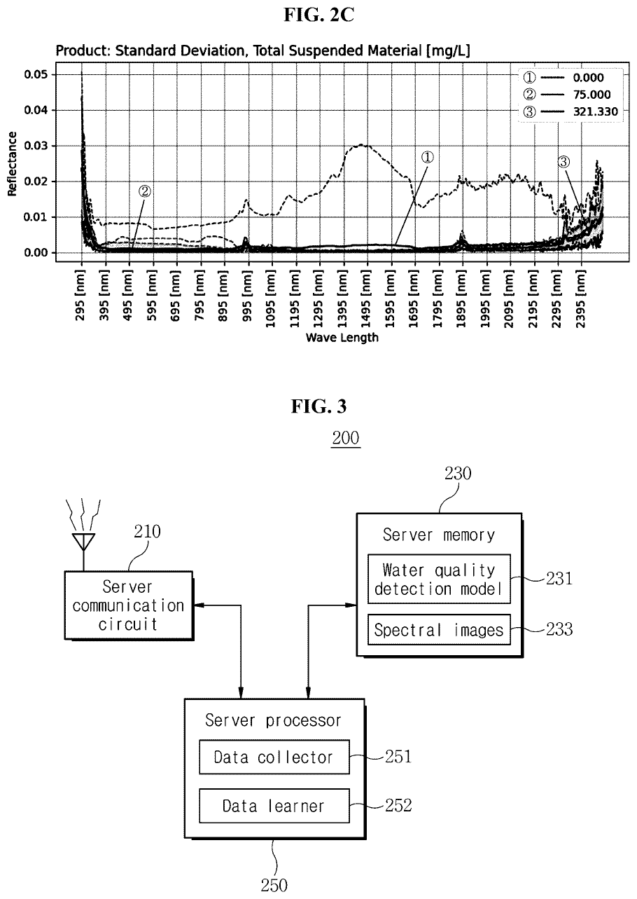

FIG. 3 is a diagram showing an example of components of a main server device according to the first embodiment of the present invention. As described above, if the water quality management function of the present invention is designed to be performed solely by the water quality management device 100, the main server device 200 may be omitted.

Referring to FIG. 3, the main server device 200 of the present invention may include a server communication circuit 210, a server memory 230, and a server processor 250.

The server communication circuit 210 may form a communication channel with the water quality management device 100 through the base station 21 or directly. The server communication circuit 210 may collect a plurality of spectral images from the water quality management device 100 at a designated cycle or in response to the occurrence of a predefined event. The server communication circuit 210 may transmit information related to the generation of a water quality detection model to a designated administrator terminal 300 or institution.

The server memory 230 may store at least one program or data required for the operation of the main server device 200. For example, the server memory 230 may store a water quality detection model 231. Additionally, the server memory 230 may store a plurality of spectral images 233 (or spectral data) received from the water quality management device 100. Also, the server memory 230 may store at least one sensing information and at least one RGB image received from the water quality management device 100.

The server processor 250 may control the transmission and processing of signals required for the operation of the main server device 200, the storage of results, the transmission of results, or the transmission of messages corresponding to results. In this regard, the server processor 250 may include a data collector 251 and a data learner 252.

The data collector 251 may request a plurality of spectral images related to the stored water 50 from the water quality management device 100 at a predefined cycle or in response to the occurrence of a predefined event. According to the first embodiment, the data collector 251 may collect a plurality of spectral images related to the stored water 50 for a certain period of time prior to a time for the generation of a generative model (or a water quality detection model) to be used for water quality management. In another example, the data collector 251 may request at least one of sensing information and RGB images collected together at the time of collecting the spectral images from the water quality management device 100 and store them in the server memory 230.

The data learner 252 may perform learning on a plurality of spectral images collected by the data collector 251. In this regard, the data learner 252 may perform learning on the plurality of spectral images related to the stored water 50 based on a learning algorithm stored in the server memory 230, thereby generating a water quality detection model. In this process, the data learner 252 may perform at least a part of the operation of the processor 150 described above in FIG. 2A. For example, the data learner 252 may classify the spectral images 233 stored in the server memory 230 into spectral images from which a suspended material is detected and spectral images from which a suspended material is not detected, and perform learning on each of the classified spectral images. For example, the data learner 252 may perform at least one of learning on a generative model including a suspended material and learning on a generative model including no suspended material. The data learner 252 may perform clustering on data classified according to the presence or absence of a suspended material, and store a cluster confirmed based on a clustering result (or a cluster confirmed based on a user input) and corresponding spectral images (or additionally, sensing information collected at the time of collecting the spectral images) in a spectral library.

Upon completing learning on the plurality of spectral images 233 and generating the water quality detection model 231, the data learner 252 may provide a guidance message to the administrator terminal 300 (or the water quality management device 100) that requested it. The data learner 252 may store the generated water quality detection model 231 in the server memory 230.

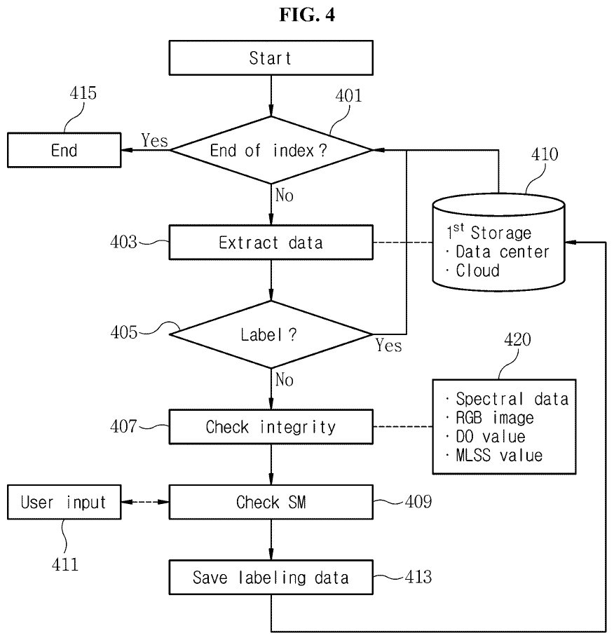

FIG. 4 is a diagram showing an example of a labeling method for training a water quality detection model according to the first embodiment of the present invention. Hereinafter, although it will be described that the water quality management device 100 trains a suspended material detection model in relation to water quality management, the present invention is not limited thereto. For example, various methods described below may be equally applied to the operation of the server processor 250 of the main server device 200.

Referring to FIG. 4, in the labeling method related to the water quality detection model (or suspended material detection model) among methods of operating the water quality management device 100 according to the first embodiment of the present invention, the processor 150 of the water quality management device 100 may check whether the index value of a currently collected spectral image is the last index value in step 401 (end of index?). In this regard, the processor 150 of the water quality management device 100 may collect a plurality of spectral images to be used for labeling at a certain cycle or based on the occurrence of various events. For example, the water quality management device 100 may collect spectral images at a time when at least one of the following occurs: a change in the water 50 stored in a tank (e.g., new water flows in, at least part of the water stored in the tank is discharged, or the flow of the stored water 50 is changed) or a change in the environment surrounding the tank (e.g., a change in the surrounding weather-a change in at least one of conditions where the external air temperature or water temperature is below or above a certain temperature, conditions where the external wind speed is above a certain value, conditions where the external wind direction is in a certain direction, and conditions where the external illuminance is above a certain value). Alternatively, the processor 150 of the water quality management device 100 may collect spectral images at a predefined regular cycle or in real time. The processor 150 of the water quality management device 100 may set a predefined number of indexes for labeling the collected spectral images. Thereafter, the water quality management device 100 may perform operation 401 (e.g., an index check operation).

If the index of the current spectral image is not the last index, the processor 150 may perform data extraction (e.g., the current spectral image acquired from a first storage 410) in step 403. In relation to the data extraction, the processor 150 may acquire the spectral image stored in the first storage 410. The first storage 410 may be, for example, the memory 130 described above in FIG. 2, or a data center or cloud.

In step 405, the processor 150 may check whether labeling is applied. If there is a label in step 405, the processor 150 may return to step 401 and re-perform the subsequent operations.

On the other hand, if there is no label in the spectral image corresponding to the index to be currently processed, the processor 150 may perform an integrity check in step 407. In relation to the integrity check, the processor 150 may acquire at least one of the spectral data (or spectral image), RGB image, DO value, and MLSS value related to the current index from a second storage 420. The second storage 420 may be, for example, the same storage as the first storage 410 or the memory 130 (or data center, cloud, etc.) described above in FIG. 2. If it is determined that there is no integrity at the result of integrity check, the processor 150 may process the spectral image of the current index as an error, return to step 401, and re-perform the subsequent operations.

If it is determined that there is the integrity through the integrity check, the processor 150 may perform a suspended material (SM) check on the spectral image corresponding to the current index in step 409. In relation to the SM check, the processor 150 may output in step 411 a user interface (UI) for a user input (human interaction) to the display 170 having an input function. The user can check the spectral image (or at least some values of the RGB image, DO value, and MLSS value collected at the time when the spectral image is collected) through the display 170 and perform an input of determining whether or not a suspended material exists. In this operation, the processor 150 may output at least some of a reference spectral image, reference RGB image, reference DO value, and reference MLSS value when a suspended material exists to the display 170.

In step 413, the processor 150 may generate labeling data for the spectral image of the current index including the result of the suspended material check in response to the user input, and may store the generated labeling data in the first storage 410. After the data is stored in the first storage 410, the processor 150 may increase the index value, and then return to step 401 to re-perform the subsequent operations. If the current index is the last index in step 401, the processor 150 may complete the labeling operation in step 415. Meanwhile, the last index may be a specific value (e.g., a null value indicating the last index) in which no separate data (e.g., a spectral image) is entered.

As described above, for the spectral images, the processor 150 may perform labeling on data including a suspended material and data including no suspended material.

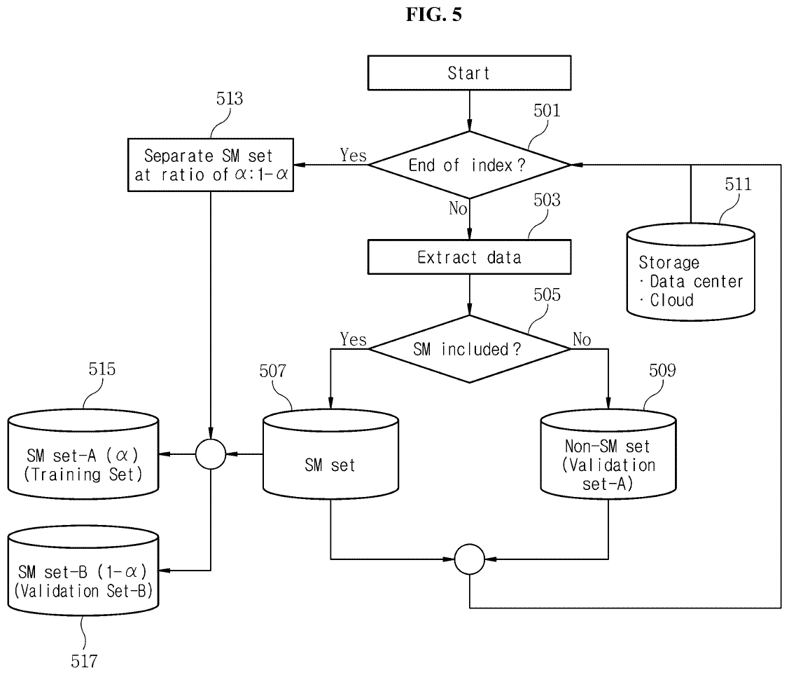

FIG. 5 is a diagram showing an example of a learning preparation method for a water quality detection model according to the first embodiment of the present invention. The learning preparation method for the water quality detection model described in FIG. 5 may be equally applied to the operation of the server processor 250 in the main server device 200 as well as the water quality management device 100.

Referring to FIG. 5, the learning preparation method among the operating methods of the water quality management device 100 according to the first embodiment of the present invention may include, in step 501, an operation of checking whether the index currently being processed by the processor 150 of the water quality management device 100 is the last index. The index may be assigned to spectral images collected for learning or, as described above in FIG. 4, assigned to labeling data for model generation.

If the index currently being processed is not the last index, in step 503, the processor 150 may perform a data extraction (or collection, acquisition) operation (an operation of acquiring a spectral image corresponding to the current index from a storage). In relation to the index, the processor 150 may identify the index of the labeling data described above in FIG. 4. For example, the labeling data assigned the last index may be a specific value that does not include separate data, and a value (e.g., null) indicating the last index may be entered.

In step 505, the processor 150 may check whether the acquired data (e.g., the spectral image of the current index) includes a suspended material (SM). If the acquired data (or the spectral image corresponding to the currently processed index) includes a suspended material, the processor 150 may store the acquired data in a suspended material containing set (SM set) in step 507. If the acquired data (or the spectral image corresponding to the currently processed index) does not include a suspended material, the processor 150 may store the acquired data in a non-suspended material containing set (Non-SM set, validation set-A) in step 509. Thereafter, the processor 150 may store the SM data set and the Non-SM data set in a designated storage (e.g., a data center or a cloud) (or the first storage 410 or the memory 130 described above) in step 511.

Next, the processor 150 may increase the count of the index value and, in step 501, check whether the index value corresponding to the increased count is the last index value. If the index value to be processed is the last index value, the processor 150 may separate the suspended material data set (SM set) at a predefined ratio of a:1−a in step 513. Here, valid data may not be separately defined for the last index, and a predefined last value (e.g., null) may be defined. The above ratio of a:1−a may indicate, for example, a ratio (a) for training and a ratio (1−a) for validation. For example, the training ratio (a) and the validation ratio (1−a) may be 2:8 or 3:7, and the above ratio may be changed by a user (or administrator).

When the training and validation ratios of the suspended material data set (SM set) are determined, the processor 150 may store the training suspended material data set (SM set-A (ratio of a), training set) together with the suspended material data set (SM set) in a predefined storage (e.g., the memory 130) in step 515, and store the validation suspended material data set (SM set-B (ratio of 1−a), validation set-B) together with the suspended material data set (SM set).

As described above, the processor 150 of the water quality management device 100 can classify the entire data according to whether a suspended material is included, and it can separate the data including the suspended material at a predefined ratio to store and manage as a training data set and a validation data set.

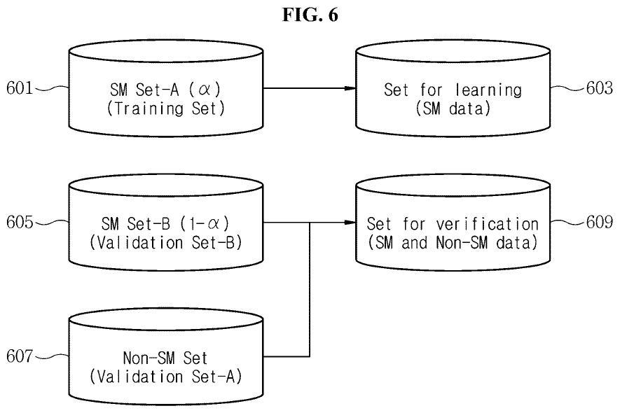

FIG. 6 is a diagram showing another example of a learning preparation method for a water quality detection model according to the first embodiment of the present invention. The learning preparation method for the water quality detection model described in FIG. 6 may be equally applied to the operation of the server processor 250 in the main server device 200 as well as the water quality management device 100.

Referring to FIG. 6, another example of the learning preparation method among the operating methods of the water quality management device 100 according to the first embodiment of the present invention may include an operation in which the processor 150 of the water quality management device 100 extracts a training suspended material data set (SM set-A (a)) stored in a storage (e.g., the memory 130) in step 601 and converts the extracted training suspended material data set into a training data set (training set, SM data) in step 603. The training suspended material data set (SM set-A (a)) may correspond to a data set of ratio (a) in the entire suspended material data set, as described above in FIG. 5, and may be, for example, a data set corresponding to 80% of the entire suspended material data set. In this case, the processor 150 may convert the 80% suspended material data set into a training data set.

Meanwhile, the processor 150 may collect a validation suspended material data set (SM set-B (1−a), validation set-B) in step 605, collect a non-suspended material data set (Non-SM set, validation set-A) in step 607, and then, in step 609, integrate the validation suspended material data set and the non-suspended material data set to convert them into a validation data set (validation set, SM and Non-SM data). The validation suspended material data set may include the suspended material data set of the remaining ratio (e.g., 1−a) excluding the training suspended material data set from the entire suspended material data set, as described above in FIG. 5. For example, the remaining ratio may be 20%, and in this case, the 20% validation data set may be used later for the validation of the generative model.

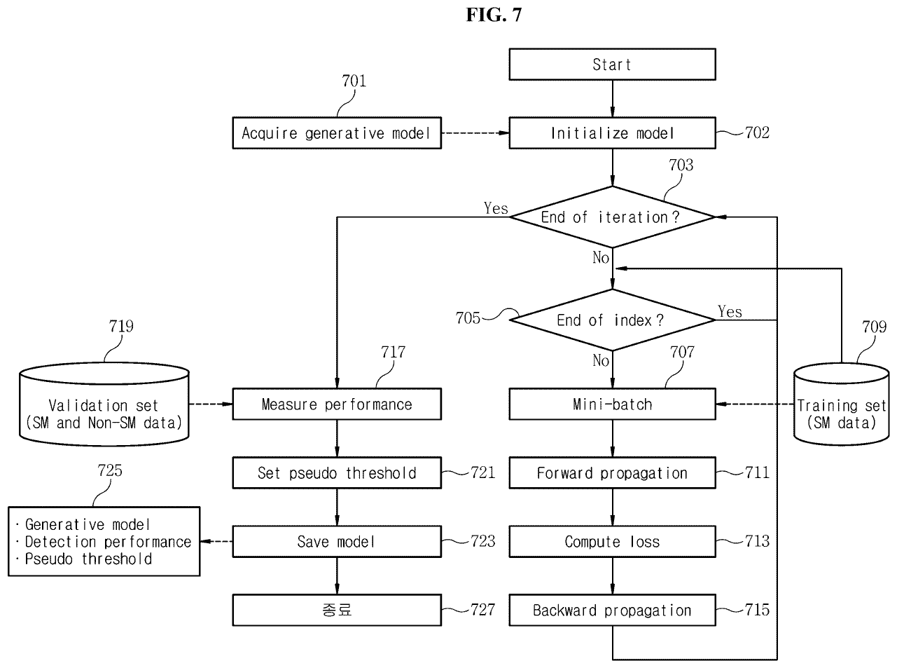

FIG. 7 is a diagram showing an example of a learning method for a water quality detection model according to the first embodiment of the present invention. The learning method for the water quality detection model described in FIG. 7 may be equally applied to the operation of the server processor 250 in the main server device 200 as well as the water quality management device 100.

Referring to FIG. 7, the processor 150 of the water quality management device 100 may acquire a generative model (or water quality detection model) from a storage (e.g., the memory 130) in step 701, and then perform model initialization in step 702.

Thereafter, in step 703, the processor 150 may check whether a current processing operation is the last step of an iteration operation (end of iteration). If the current processing operation is not the last step of the iteration operation, the processor 150 may check in step 705 whether an index to be currently processed is the last index (end of index). The last index may have a value indicating, for example, that the index is the last index. If the index currently being processed is the last index, the processor 150 may return to step 703 and re-perform the operation.

If the current index value to be processed is not the last index value, the processor 150 may perform a mini-batch operation in step 707. In this operation, the processor 150 may acquire training data having a predefined batch size (e.g., within a size defined in the mini-batch) for one iteration from the corresponding storage (e.g., the memory 130) in step 709. The storage may transfer the training data set to the processor 150 in step 709, and also provide information about the current training data set (e.g., iteration and index information) to the processor 150 in step 705.

When data loading (mini-batch) is completed, the processor 150 may perform training of the corresponding iteration by going through steps 711 to 715 (e.g., forward propagation, compute loss, backward propagation). After learning is completed, the processor 150 may return to step 703 and re-perform the subsequent operations.

On the other hand, in step 703, if the current iteration step is the last iteration step, the processor 150 may perform a performance evaluation (measure performance) in step 717. In this regard, the processor 150 may acquire a validation data set (e.g., SM and non-SM data) from the storage (e.g., the memory 130) in step 719. The processor 150 may input the validation data set into the generated model and thereby perform an accuracy evaluation on the presence or not of a suspended material. In the performance evaluation process, if a performance evaluation result lower than a predefined threshold value is obtained, the processor 150 of the water quality management device 100 may further collect a predefined amount of spectral images and re-perform the operations described in FIGS. 4 to 7 for the collected spectral images.

In step 721, the processor 150 may set a pseudo threshold value according to the value of the accuracy evaluation. In step 723, the processor 150 may save the model with the pseudo threshold value set. Accordingly, as in step 725, the generative model, detection performance, and pseudo threshold value may be stored in the storage. Thereafter, in step 727, the processor 150 may end the model generation operation.

### Second Embodiment

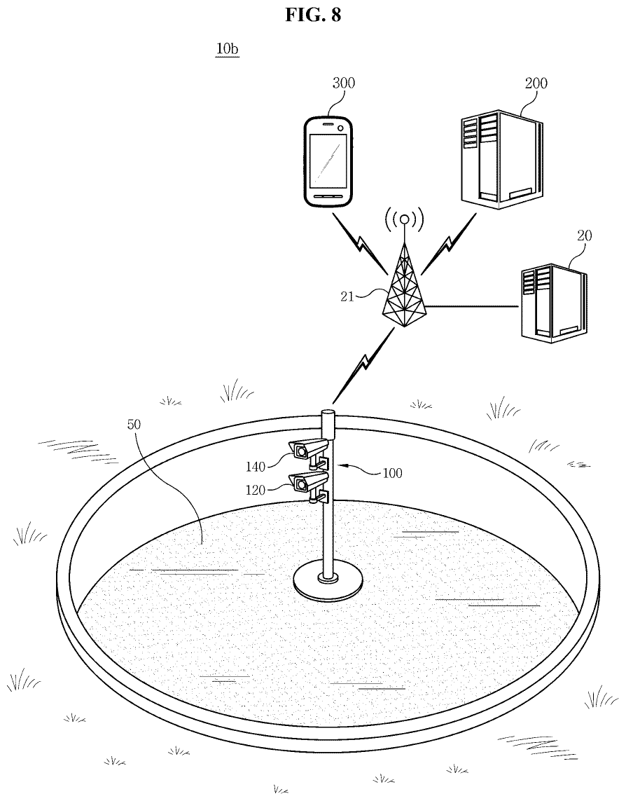

FIG. 8 is a diagram showing an example of a water quality management system according to the second embodiment of the present invention.

Referring to FIG. 8, the water quality management system 10b of the present invention may include stored water 50 (e.g., water stored in a tank), a water quality management device 100, a base station 21, a relay server device 20, a main server device 200, and an administrator terminal 300. Here, it is exemplified that the water quality management system 10b includes the base station 21 for communication between the water quality management device 100 and the relay server device 20, the main server device 200, or the administrator terminal 300, but the present invention is not limited thereto. For example, without the base station 21, the water quality management device 100 and the main server device 200 or the water quality management device 100 and the relay server device 20 may also be directly connected via a wired cable or a short-range wireless communication scheme. In this case, the base station 21 may be omitted from the water quality management system 10b. In addition, although it is exemplified that the relay server device 20 is included in the water quality management system 10b, the present invention is not limited thereto. For example, the relay server device 20 may be omitted or provided as being integrated with the main server device 200.

The stored water 50 may be water stored in a tank having a certain size. The tank that stores water may be at least one of various tank types such as a sewage tank of a sewage treatment plant or a water supply tank for water supply treatment. Therefore, the stored water 50 may be water stored in a sewage tank, water stored in a water supply tank, or the like. The stored water 50 in the present invention may include a suspended material, for example, depending on various external or internal conditions of the water tank. The stored water 50 including a suspended material may be purified through a purification process. The stored water 50 may be located within a shooting angle at which a spectral camera 120 and an RGB camera 140 disposed in the water quality management device 100 can photograph. In addition, the stored water 50 may be positioned so that it can be sensed by at least one sensor disposed in the water quality management device 100 or independently disposed at a certain place of the water tank.

The water quality management device 100 may include the spectral camera 120 that collects spectral images of the stored water 50, the RGB camera 140 that collects RGB images of the stored water 50, and a mounting structure that mounts the spectral camera 120 and the RGB camera 140. Although it is described that the water quality management device 100 includes both the spectral camera 120 and the RGB camera 140, the present invention is not limited thereto. For example, the water quality management device 100 may include only the spectral camera 120. Additionally, the water quality management device 100 according to the second embodiment of the present invention may further include at least one sensor that can collect sensing information related to the water quality of the stored water 50 in addition to the spectral images. The water quality management device 100 may detect the water quality of the stored water 50 based on at least some of the spectral image collected by the spectral camera 120, the RGB image collected by the RGB camera 140, and the sensing information collected by the at least one sensor. In this regard, the water quality management device 100 trains a model related to water quality measurement based on collected data (e.g., the spectral images collected by the spectral camera 120, at least some of the RGB image and sensing information collected together at the time of collecting the spectral images).

Meanwhile, the water quality management device 100 may be configured to transmit the collected data (e.g., at least some of the spectral image collected by the spectral camera 120, the RGB image collected by the RGB camera 140, and the sensing information collected by the at least one sensor) related to the stored water 50 to the main server device 200. In this case, the water quality management device 100 may form a communication channel with (or may be directly connected to) the main server device 200 through the base station 21. The water quality management device 100 may transmit the collected data (e.g., at least some of the spectral image collected by the spectral camera 120, the RGB image collected by the RGB camera 140, and the sensing information collected by the at least one sensor) to the main server device 200.

The base station 21 may be spaced apart from the water quality management device 100 by a certain distance and disposed geographically at a distance or location where a wireless communication channel can be formed with the water quality management device 100. Alternatively, the base station 21 may be connected to the water quality management device 100 by wire. Additionally, the base station 21 may support forming at least one of communication channels between the water quality management device 100 and the main server device 200, between the water quality management device 100 and the relay server device 20, between the relay server device 20 and the main server device 200, between the water quality management device 100 and the administrator terminal 300, and between the main server device 200 and the administrator terminal 300.

The main server device 200 may form a communication channel with the water quality management device 100 through the base station 21 (or directly without the base station 21). The main server device 200 may receive the collected data (e.g., at least some of the spectral image collected by the spectral camera 120, the RGB image collected by the RGB camera 140, and the sensing information collected by the at least one sensor) from the water quality management device 100 and perform the learning of a water quality detection model based on the received data. Thereafter, the main server device 200 may perform spectral image analysis on the stored water 50 at a specific time based on the learning-completed water quality detection model, thereby estimating the water quality of the stored water 50. The main server device 200 may transmit the water quality result (or water quality estimation result) of the stored water 50 to a designated administrator terminal 300 (or user terminal) or output it through a connected display device. In this operation, the main server device 200 may produce whether the stored water 50 contains a suspended material, the size of the suspended material, the degree of the suspended material, whether water purification (or purification) work is needed due to the suspended material, etc., and transmit the produced information to the administrator terminal 300. Meanwhile, if the learning of the water quality detection model and the estimation of the water quality result of the stored water 50 are designed to be processed in the water quality management device 100, the main server device 200 may be omitted. In this case, the water quality management device 100 may directly produce the water quality result and transmit it to the administrator terminal 300 or output it to a connected display.

Although it is described above that at least one sensor is included in the water quality management device 100, the present invention is not limited thereto. For example, the at least one sensor may be placed at a specific location of the stored water 50 without being included in the water quality management device 100, have a communication module on its own, and be connected to the relay server device 20 via the base station 21. Here, the communication module included in the at least one sensor may be connected to the relay server device 20 via a wired cable, or may be formed as a wireless communication module and connected to the relay server device 20 via the base station 21. The at least one sensor may collect sensing information on the stored water 50 according to predefined schedule information, and transmit the collected sensing information to the relay server device 20.

The relay server device 20 may be connected to at least one sensor, and transmit sensing information collected from the at least one sensor to at least one of the water quality management device 100 and the main server device 200 at regular intervals or in real time. Alternatively, the relay server device 20 may receive control information regarding at least one sensor from at least one of the water quality management device 100 and the main server device 200, and control the at least one sensor based on the received control information to collect sensing information. Meanwhile, as mentioned above, the relay server device 20 may be integrated with the main server device 200 or, if at least one sensor is included in the water quality management device 100, may be omitted.

The administrator terminal 300 may form a communication channel with at least one of the water quality management device 100, the relay server device 20, and the main server device 200 through the base station 21. The administrator terminal 300 may receive the estimated water quality result from at least one device. The administrator terminal 300 may output the water quality result through an output device (e.g., a display or an audio device). In another example, the administrator terminal 300 may receive a water purification operation request message according to the water quality result from at least one device. The administrator terminal 300 may output the received water purification operation request message. Alternatively, the administrator terminal 300 may output the water quality result and the water purification operation request message simultaneously to provide information so that the administrator can determine when to perform the water purification operation on the stored water 50. In an example, the administrator terminal 300 may be a terminal of an administrator who manages a water purification device capable of performing the water purification operation on the stored water 50. In this regard, the water quality management device 100 may further include a water purification device capable of performing water purification on the stored water 50, and the administrator terminal 300 may include the water purification device.

As described above, the water quality management system 10b according to the second embodiment of the present invention can acquire spectral images of the stored water 50, generate a water quality detection model based on learning about the acquired spectral images, and support the estimation of water quality results at a specific time based on the model. In an example, the water quality management system 10b of the present invention can collect information about the presence or absence of a suspended material having a significant impact on water quality, the shape and extent of the suspended material, and construct and operate the water quality detection model related to the suspended material, thereby effectively supporting water quality management.

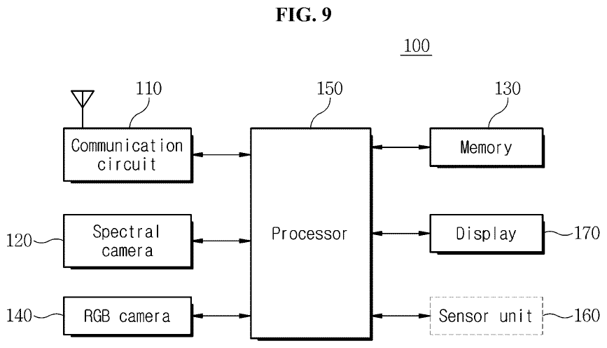

FIG. 9 is a diagram showing an example of components of a water quality management device according to the second embodiment of the present invention. In the following description, a form in which the relay server device 20 is integrated with the main server device 200 is exemplified. However, the present invention is not limited thereto, and the relay server device 20 may be separately included in the water quality management system 10b for the operation of a sensor unit 160 including at least one sensor.

Referring to FIG. 9, the water quality management device 100 according to the second embodiment of the present invention may include a communication circuit 110, a spectral camera 120, a memory 130, an RGB camera 140, a sensor unit 160, a display 170, and a processor 150. Here, if the water quality management device 100 is configured to perform a water quality management by using only spectral images, at least one of the RGB camera 140 and the sensor unit 160 may be omitted. In addition, the water quality management device 100 may further include a mounting structure for mounting the spectral camera 120 so that the spectral camera 120 can be oriented to an angle capable of capturing at least a portion of the stored water 50. In addition, the water quality management device 100 may further include a power supply (e.g., a permanent power supply or a battery) required for the operation of at least one of the above-described components, for example, the communication circuit 110, the spectral camera 120, the memory 130, the RGB camera 140, the sensor unit 160, the display 170, and the processor 150. Meanwhile, at least one of the components of the water quality management device 100 may be omitted.

The communication circuit 110 may form a communication channel with the main server device 200 (or the relay server device 20 or the administrator terminal 300) via the base station 21. Alternatively, the communication circuit 110 may form a communication channel directly with the main server device 200 or the relay server device 20 without going through the base station 21. If the main server device 200 is designed to perform the water quality management according to the second embodiment of the present invention, the communication circuit 110 may transmit spectral images collected by the spectral camera 120 (additionally at least some of RGB images and sensing information) to the main server device 200. On the other hand, a water quality management function may be performed independently by the water quality management device 100. In this case, the communication circuit 110 may transmit a warning message (e.g., a message requesting water purification work for the stored water 50) based on the water quality result to the main server device 200. Alternatively, the communication circuit 110 may transmit at least some of the water quality result and the warning message to a resident of a certain area or an administrator of a certain institution (e.g., the administrator terminal 300) under the control of the processor 150. The communication circuit 110 may also receive an artificial neural network algorithm required for fluid path diffusion prediction from the main server device 200.

The spectral camera 120 may be disposed to capture a spectral image for at least a section (or at least a point) of the stored water 50. The spectral camera 120 may capture the spectral image of the stored water 50 under the control of the processor 150. For example, when a predefined scheduled event occurs, the spectral camera 120 may capture a plurality of spectral images related to the stored water 50 in response to the occurrence of the event. For example, under the control of the processor 150 (or in response to a request of the main server device 200), the spectral camera 120 may acquire the plurality of spectral images related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). Alternatively, the spectral camera 120 may capture the plurality of spectral images related to the stored water 50 according to a predefined regular cycle.

The memory 130 may store at least one program or data required for the operation of the water quality management device 100. For example, the memory 130 may store at least some of a control program required for operating the spectral camera 120, a plurality of spectral images acquired by the spectral camera 120, a water quality detection model generated based on the plurality of spectral images, a spectral image acquired at the current time and a water quality result derived therefrom, a warning or notifying message corresponding to the water quality result. In addition, the memory 130 may store an artificial neural network algorithm or a machine learning algorithm required for the water quality detection model.

The RGB camera 140 may be disposed to have the same shooting angle or face the same shooting point as the shooting angle or shooting point of the spectral camera 120. The RGB camera 140 may acquire at least one RGB image of the stored water 50 while being synchronized with the time at which the spectral camera 120 captures a spectral image. For example, the RGB camera 140 may acquire at least one RGB image related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). The acquired RGB image may be temporarily stored in the memory 130 and, in response to the control of the processor 150, may be transmitted to a designated device (e.g., the main server device 200). For example, the acquired RGB image may be output through the display 170 of the water quality management device 100.

The sensor unit 160 may be disposed in the water quality management device 100 and collect sensor information related to the water quality of the stored water 50 at the time when the spectral camera 120 acquires the spectral image. For example, the sensor unit 160 may include at least one of a dissolved oxygen (DO) sensor, a mixed liquor suspended solids (MLSS) sensor, a biochemical oxygen demand (BOD) sensor, and a chemical oxygen demand (COD) sensor, which can directly measure water quality information. In addition, the sensing information collected by the sensor unit 160 may be temporarily stored in the memory 130 and, depending on the design form, may be transmitted to the main server device 200. Meanwhile, at least one sensor included in the sensor unit 160 may be connected to the relay server device 20 and configured to collect sensing information in response to the control of the relay server device 20 and then transmit the sensing information to the water quality management device 100 or the main server device 200. The at least one sensing information collected by the sensor unit 160 may be used for at least one operation of water quality detection model generation and water quality result estimation. The sensor unit 160 may be configured to collect the sensing information at the time of spectral image acquisition. Alternatively, the sensor unit 160 may be configured to collect the sensing information at a predefined time or cycle or in real time.

The display 170 may output various information screens required for the operation of the water quality management device 100. For example, the display 170 may output a screen related to the operation of at least one of the spectral camera 120 and the RGB camera 140 of the water quality management device 100. The display 170 may output at least in part the operating information of the sensor unit 160 and the sensing information collected by the sensor unit 160. In addition, the display 170 may output a screen related to learning of a generative model (or a water quality detection model). The display 170 may include a touch screen that supports a user input function. The display 170 may output at least one of the water quality result of the currently collected spectral image and the warning message corresponding to the water quality result under the control of the processor 150.

The processor 150 may control the spectral camera 120 to acquire a plurality of spectral images or a current spectral image related to the stored water 50 in response to a predefined event or a request from the main server device 200 or the administrator terminal 300. The processor 150 may perform at least one of an operation of generating the water quality detection model based on the plurality of acquired spectral images and an operation of estimating the water quality result of the currently collected spectral image.

For example, the processor 150 may generate a water quality detection model based on a suspended material. The processor 150 may collect spectral images of the stored water 50 by using the spectral camera 120 and may generate a model capable of determining the presence or absence of a suspended material (SM) by analyzing the collected spectral images. For example, in the process of analyzing the acquired spectral image, the processor 150 may determine that a suspended material exists in the spectral image if the wavelength is relatively short compared to predefined criteria and the fluctuation range of reflectance is less than the predefined criteria when observing the situation. The processor 150 may determine that a suspended material does not exist in the spectral image if the wavelength is relatively long compared to the predefined criteria, the overall reflectance is high, and the variation range is greater than the predefined criteria. The predefined criteria may be the average value of the acquired spectral images or may be defined by the setting value of the administrator involved in model generation. For example, the consistency of the spectral images in the case where a suspended material exists may be higher than the consistency of the spectral images in the case where a suspended material does not exist. The processor 150 may use the high-consistent spectral images with suspended materials for learning of a generative model.

In relation to the generative model learning (or water quality detection model learning), the processor 150 may use the Auto-Encoder or the Generative Adversarial Network among artificial neural networks for deep learning. The processor 150 may use a traditional machine learning model in addition to a deep learning algorithm, and the present invention is not limited to deep learning and machine learning models. The processor 150 may output a user UI (e.g., a screen displaying a list of training data, a screen for selecting at least one item included in the list) through the display 170 so that a user (or an administrator involved in generative model learning) can manually designate a list of training data. The processor 150 may process label designation for each data sample in response to a user input. In the labeling operation, the processor 150 may include spectral data (or spectral image) in the label, and may also output at least one type of sensing information collected by the sensor unit 160 to the display 170 so that the user can refer to it during the labeling process.

In another example, the processor 150 may support semi-automatic designation of a label by a criterion allowed by the user. In applying the criterion, the processor 150 may include a spectral image (or spectral data) in the corresponding labeled data and output at least part of the sensing information collected by the sensor unit 160 to the display 170 for reference. In relation to the semi-automatic designation, the processor 150 may output to the display 170 a screen that allows the user to designate a specific criterion value (e.g., values obtained by multiplying the average value and the standard deviation of frequency values of spectral images by a predefined coefficient value) for determining the presence or not of a suspended material, and determine the specific criterion value for determining the presence or not of a suspended material in the spectral image by a user input. When the criterion value is determined, the processor 150 may analyze the spectrum of spectral images to check whether frequency distribution is less than or greater than the criterion value, and thereby automatically determine the presence or not of a suspended material.

The processor 150 may input a spectral image into the learning-completed generative model to determine whether a suspended material exists. If a restoration error is greater than a predefined threshold value, the processor 150 may consider or determine this to be a situation that has not been observed during the learning process and in which no suspended material exists. If the restoration error is less than the predefined threshold value, the processor 150 may consider this as a situation that has been observed (or observed similarly) during the learning process and determine that a suspended material exists. In an example, the determination criterion may use a threshold at a point where the classification performance index (e.g., F1-score) is maximum during the learning process. Here, the threshold may be adjusted or designated by a user input.

In relation to a semi-automatic spectral library construction through a suspended material discrimination model, if the processor 150 determines that a suspended material exists in the spectral image according to the suspended material discrimination method described above, the processor 150 may store the spectral image determined to have a suspended material in a suspended material data archive area (or a separate suspended material archive) of the memory 130. If the processor 150 determines that a suspended material does not exist, the processor 150 may store the spectral image in the non-suspended material (non-SM) data archive area (or non-suspended material data archive) of the memory 130. If the number of data held by the suspended material archive exceeds a predefined certain amount or a user request occurs, the processor 150 may cluster the data stored in the archive by similar characteristics. An example of a clustering method may be K-means clustering, but the present invention is not limited thereto. Based on settings or user input, the processor 150 may perform the same clustering process on data stored in the non-suspended material (non-SM) data archive. When clustering is completed, the processor 150 may output the clustering result to the display 170 to allow the user (or administrator) to check the clustering result, and may confirm the information indicated by the cluster in response to a user input. In this process, the information output to the display 170 may include the result of a specific cluster, a spectral image corresponding to the cluster, and/or sensing information collected together when the spectral image is collected. When the user confirms the cluster information, the processor 150 may add the confirmed cluster to the spectral library. For example, the confirmed information may include one or more of information necessary for water quality management, such as water quality status, types of suspended materials, and measures to be taken for water quality management. Based on setting or a user request, the processor 150 may support determining information by referring to other data existing in a hyperspectral library. In an example, based on setting or a user input, the processor 150 may delete or overwrite information existing in the hyperspectral library. Based on setting or a user request, the processor 150 may support clustering of at least one of the suspended material data archive and the non-suspended material data archive and a clustering-based water quality management function.

As an indicator of water quality status in relation to water quality management, the processor 150 may use at least one of a type of using the presence or not of a suspended material and a type of using a water quality status indicator utilizing a spectral library. The criterion for water quality management may selectively use the above-mentioned two types of model operation, and at least one of the indication of the presence or not of a suspended material or the water quality status indication based on a spectral library may be used as needed during operation. In an example, the processor 150 may first determine the presence or not of a suspended material in the stored water 50, and if a suspended material is present, may determine the current detailed status using a spectral library.

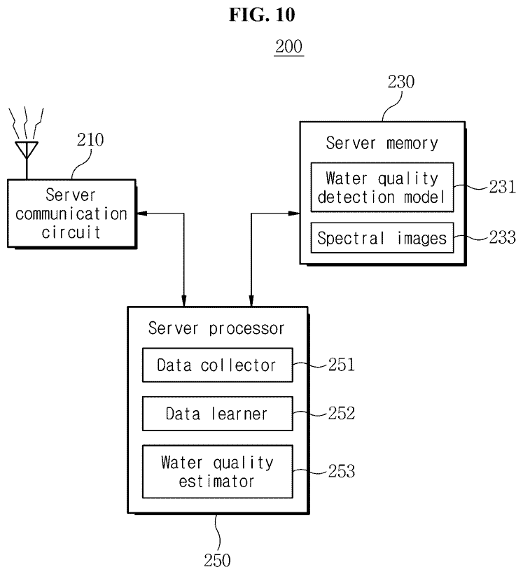

FIG. 10 is a diagram showing an example of components of a main server device according to the second embodiment of the present invention. As described above, if the water quality management function of the present invention is designed to be performed solely by the water quality management device 100, the main server device 200 may be omitted.

Referring to FIG. 10, the main server device 200 of the present invention may include a server communication circuit 210, a server memory 230, and a server processor 250.

The server communication circuit 210 may form a communication channel with the water quality management device 100 through the base station 21 or directly. The server communication circuit 210 may collect a plurality of spectral images and a current spectral image from the water quality management device 100 at a designated cycle or in response to the occurrence of a predefined event. The server communication circuit 210 may transmit a water quality result and a corresponding warning message to a designated administrator terminal 300 or institution.

The server memory 230 may store at least one program or data required for the operation of the main server device 200. For example, the server memory 230 may store a water quality detection model 231. Additionally, the server memory 230 may store a plurality of spectral images 233 (or spectral data) received from the water quality management device 100. Also, the server memory 230 may store at least one sensing information received from the water quality management device 100. For example, the server memory 230 may store the plurality of spectral images 233 received from the water quality management device 100 and used to generate the water quality detection model 231. The server memory 230 may store the water quality detection model 231 generated based on the plurality of spectral images 233. In addition, the server memory 230 may also store a current spectral image used for a current water quality check.

The server processor 250 may control the transmission and processing of signals required for the operation of the main server device 200, the storage of results, the transmission of results, or the transmission of messages corresponding to results. In this regard, the server processor 250 may include a data collector 251, a data learner 252, and a water quality estimator 253.

The data collector 251 may request a plurality of spectral images related to the stored water 50 from the water quality management device 100 at a predefined cycle or in response to the occurrence of a predefined event. According to the second embodiment, the data collector 251 may collect a plurality of spectral images related to the stored water 50 for a certain period of time prior to a time for the generation of a generative model (or a water quality detection model) to be used for water quality management. In another example, the data collector 251 may request at least some of sensing information and RGB images collected together at the time of collecting the spectral images from the water quality management device 100 and store them in the server memory 230.

The data learner 252 may perform learning on a plurality of spectral images collected by the data collector 251. In this regard, the data learner 252 may perform learning on the plurality of spectral images related to the stored water 50 based on a learning algorithm stored in the server memory 230, thereby generating a water quality detection model. In this process, the data learner 252 may perform at least a part of the operation of the processor 150 described above in FIG. 9. For example, the data learner 252 may classify the spectral images 233 stored in the server memory 230 into spectral images from which a suspended material is detected and spectral images from which a suspended material is not detected, and perform learning on each of the classified spectral images. For example, the data learner 252 may perform at least one of learning on a generative model including a suspended material and learning on a generative model including no suspended material. The data learner 252 may perform clustering on data classified according to the presence or absence of a suspended material, and store a cluster confirmed based on a clustering result (or a cluster confirmed based on a user input) and corresponding spectral images (or additionally, sensing information collected at the time of collecting the spectral images) in a spectral library.

Upon completing learning on the plurality of spectral images 233 and generating the water quality detection model 231, the data learner 252 may provide a guidance message to the administrator terminal 300 (or the water quality management device 100) that requested it. The data learner 252 may store the generated water quality detection model 231 in the server memory 230. For example, the data learner 252 may perform labeling of the plurality of spectral images 233, classification of the plurality of labeled spectral images 233 (e.g., classification into a suspended material data set including spectral images with a suspended material and a non-suspended material data set without a suspended material), and classification into training and validation (e.g., classification into a training data set of a certain ratio (e.g., 80%, changeable) for training the suspended material data set and a validation data set of a certain ratio (e.g., 20%, changeable) for validation), generate the water quality detection model 231 by using only the training data set, and then perform a performance check of the water quality detection model 231 by analyzing a restoration error after inputting the validation data set into the water quality detection model 231.

In relation to labeling, the data learner 252 may assign indexes to a plurality of spectral images and separate labeled spectral images and unlabeled spectral images based on the indexes. The data learner 252 may perform an integrity check (e.g., outputting at least some of sensing information, RGB images, and spectral images to the display 170 so that the user can check them, and performing an integrity check in response to a user input) on the unlabeled spectral images. The data learner 252 may perform a suspended material check (e.g., outputting at least some of sensing information, RGB images, and spectral images to the display 170 so that the user can check them, and determining whether or not there is a suspended material in response to a user input) on the spectral images that have passed the integrity check.

The water quality estimator 253 may calculate the restoration error of the currently collected spectral image by using the water quality detection model 231 (or the generative model), and estimate the water quality result of the currently collected spectral image based on the calculated restoration error. Alternatively, the water quality estimator 253 may estimate the water quality result of the currently collected spectral image by using a spectral library that stores the clustering result of spectral images. For example, the water quality estimator 253 may analyze the spectrum of the currently collected spectral image, perform clustering on the spectrum, compare the clustering result with clusters pre-stored in the spectral library (e.g., a cluster determined as a spectral image including a suspended material, a cluster determined as a spectral image including no suspended material, or clusters classified into various water quality states), and estimate the water quality result of the currently collected spectral image based on the comparison result. The water quality estimator 253 may transmit the water quality result estimation value to the administrator terminal 300 or transmit a guidance message or warning message according to the water quality result to the administrator terminal 300.

Meanwhile, the operations of the data collector 251, the data learner 252, and the water quality estimator 253 described in the server processor 250 may be configured in the processor 150 of the water quality management device 100 described in FIG. 9, and the water quality management device 100 may be configured to generate a water quality detection model, detect water quality by using the generated water quality detection model, and perform operations according to the detected water quality results. Therefore, among the operations related to the generation of the water quality detection model 231 and the water quality check in FIG. 9, parts that are not described in FIG. 10 may be supplemented and understood as described in FIG. 10, and similarly, parts of the description of FIG. 10 that are not described in FIG. 9 may be supplemented and understood as described in FIG. 9.

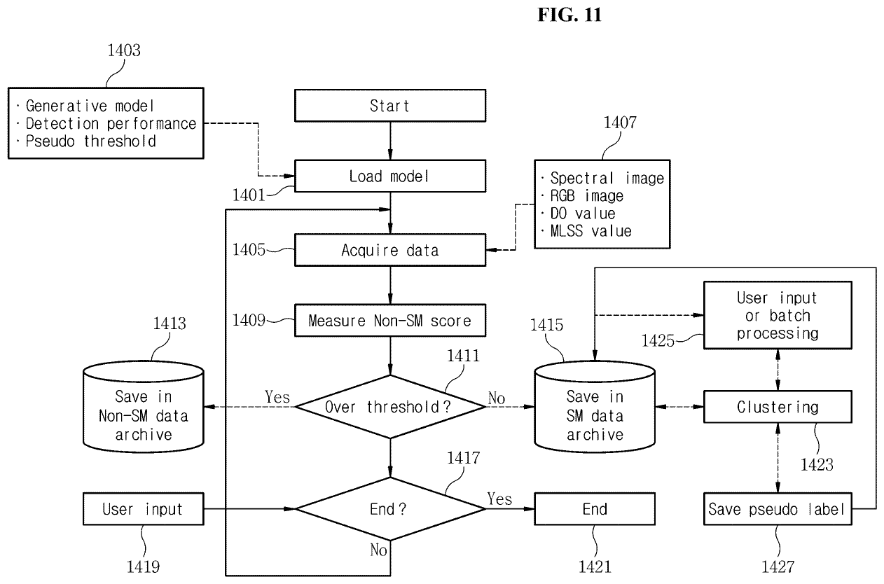

FIG. 11 is a diagram showing an example of a suspended material detection model operation according to the second embodiment of the present invention. The detection model operation described in FIG. 11 may be applied to at least one of the water quality management device 100 described in FIG. 9 and the main server device 200 described in FIG. 11. The following description is based on the operation of the processor 150 of the water quality management device 100. However, the present invention is not limited thereto, and the description may be applied equally or similarly to the operation of the server processor 250 of the main server device 200.

Referring to FIG. 11, in a water quality management method related to the operation of the suspended material detection model of the present invention, the processor 150 of the water quality management device 100 may load an initial model in block 1401 and apply a generative model, detection performance, and pseudo threshold to the loaded model in block 1403.

Afterwards, in block 1405, the processor 150 may perform acquisition of data (e.g., spectral image) stored in the memory 130. For the data acquisition, the processor 150 may read at least some of the spectral image (hyperspectral data), RGB image, and sensing information (DO value, MLSS value) from the memory 130 in block 1407.

In block 1409, the processor 150 may measure a non-suspended material (non-SM) score. The non-SM score may be calculated by analyzing a spectral image and based on the ratio of an area in which a suspended material is not included in the spectral image. For example, in relation to that calculation of the non-SM score, the water quality management device 100 may include the memory 130 that stores a spectral library including at least one of an SM data archive and a non-SM data archive. At least one of the SM data archive or the non-SM data archive may store data configured to calculate the non-SM score for the spectral image, for example. In an example, the processor 150 may determine that the water is in a clean state that does not require purification if the non-SM score is higher than a threshold value, and may determine that the water is in a state that requires purification if the non-SM score is lower than the threshold value.

In block 1411, the processor 150 may check whether the non-SM score exceeds a predefined threshold value (over threshold?). If the non-SM score exceeds the predefined threshold value, the processor 150 may store the acquired spectral image in the non-SM data archive (or a non-SM data archive area of the memory 130) in block 1413. If the non-SM score does not exceed the predefined threshold value, the processor 150 may store the acquired spectral image in the SM data archive (or an SM data archive area of the memory 130) in block 1415.

In addition, after performing a comparison between the non-SM score and the threshold value, the processor 150 may check in block 1417 whether the current process is an end process. In relation to the end process check, the processor 150 may output a screen interface related to the end process to the display 170 in block 1419 and then check whether an end-related user input (user interaction) occurs or a pre-allocated button input for end occurs. If the end-related user input occurs, the processor 150 may end the operation of the suspended material detection model in block 1421. If the end-related user input does not occur, the processor 150 may return to block 1405 and re-perform the operations of the subsequent blocks.

On the other hand, when new suspended material data (or a new spectral image including a suspended material) is stored in the SM data archive, a clustering operation for the corresponding spectral image may be performed in block 1423, user interaction or batch processing may be performed in step 1425, and pseudo label saving may be performed in step 1427.

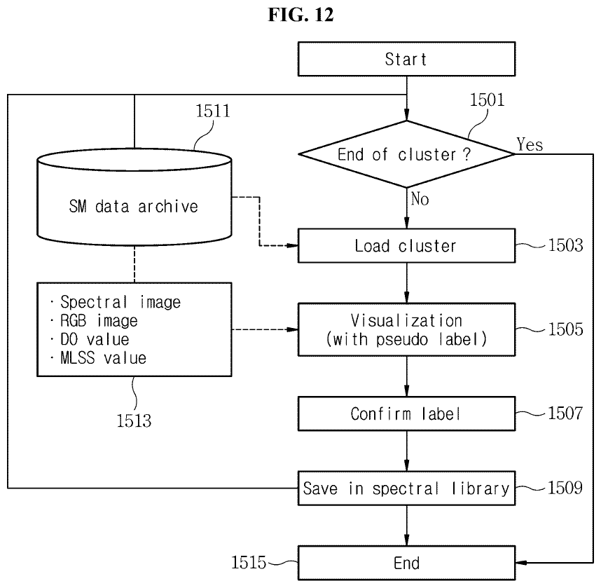

FIG. 12 is a diagram showing an example of a spectral library preparation method according to the second embodiment of the present invention. Although the method in FIG. 12 is described based on the operation of the processor of the water quality management device, the present invention is not limited thereto, and the method in FIG. 12 may correspond to at least a part of the operation of the server processor of the main server device described in FIG. 10.

Referring to FIG. 12, in a method for operating the water quality management device related to the spectral library preparation according to the second embodiment of the present invention, the processor 150 of the water quality management device 100 may check whether a cluster to be currently processed is the last cluster (end of cluster?) in step 1501. In this regard, the processor 150 may perform information processing capable of recognizing the last cluster for clusters corresponding to a plurality of spectral images. For example, the last cluster may have a predefined specific symbol or data differently from other clusters. If the cluster to be currently processed is not the last cluster, the processor 150 may load the cluster in step 1503. In this regard, the processor 150 may acquire suspended material data (or a spectral image, or cluster generated by performing clustering on a spectral image) from a suspended material data archive 1511.

In step 1505, the processor 150 may perform visualization for the loaded cluster. In this operation, the processor 150 may output a pseudo label of the loaded cluster together to the display 170, and output at least some of the spectral data (hyperspectral data), RGB image, and sensing information (DO value, MLSS value) stored in a storage 1513 (or the memory 130) to the display 170. Through the visualization process, the processor 150 may output a clustering result (e.g., a graph) corresponding to the spectral image to the display 170.

In step 1507, the processor 150 may perform label confirmation. For example, if the processor 150 determines that the data output through visualization is data including a suspended material, the processor 150 may confirm the label. In step 1509, the processor 150 may store the labeled cluster in the spectral library (save in hyperspectral library) and then return to step 1501. In this operation, the processor 150 may update a spectral library area of the suspended material data archive.

In step 1501, if it is the last cluster, the processor 150 may end the spectral library preparation operation. Here, information indicating the last cluster may not include actual cluster information separately, but may include a predefined specific symbol or data indicating the last cluster.

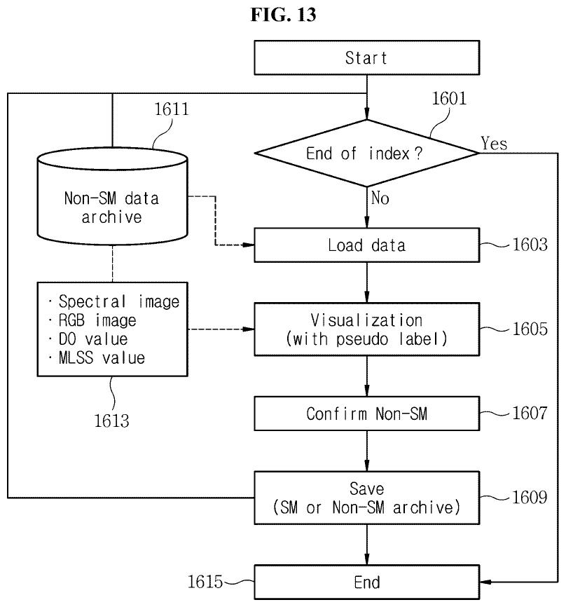

FIG. 13 is a diagram showing another example of a spectral library preparation method according to the second embodiment of the present invention. Although the method in FIG. 13 is described based on the operation of the processor of the water quality management device, the present invention is not limited thereto, and the method in FIG. 13 may correspond to at least a part of the operation of the server processor of the main server device described in FIG. 10.

Referring to FIG. 13, in relation to another example of a method for operating the water quality management device related to the spectral library preparation according to the second embodiment of the present invention, the processor 150 of the water quality management device 100 may check whether an index of information to be currently processed is the last index (end of index?) in step 1601. In this regard, the processor 150 may assign indexes to a plurality of spectral images and check whether the index corresponding to the spectral image to be currently processed is the last index. If the index to be currently processed is not the last index, the processor 150 may load data in step 1603. In this regard, the processor 150 may acquire and load non-suspended material data (or spectral image) from a non-suspended material data archive 1611.

In step 1605, the processor 150 may perform visualization for the loaded data (e.g., spectral image). In this operation, the processor 150 may output a pseudo label of the loaded data together to the display 170, and output at least some of the spectral data (hyperspectral data), RGB image, and sensing information (DO value, MLSS value) stored in a storage 1613 (or the memory 130) to the display 170. Through the visualization process, the processor 150 supports checking on the display 170 whether a non-suspended material corresponding to a spectral image is included.

In step 1607, the processor 150 may perform non-SM confirmation. For example, if the processor 150 determines that the data output through visualization is non-SM data including no suspended material, the processor 150 may confirm the currently processed data as non-SM data. Alternatively, if the processor 150 determines that the data output through visualization is SM data including a suspended material, the processor 150 may confirm the currently processed data as SM data. The SM data confirmation may be based on a user input.

In step 1609, the processor 150 may store the confirmed cluster in a designated archive (save SM or non-SM archive) and then return to step 501. In this operation, the processor 150 may store the non-SM data in a non-SM data archive 1611. In step 1601, if the index to be currently processed is the last index, the processor 150 may end the spectral library preparation operation.

Meanwhile, although the spectral library generation method is described based on the suspended material data archive and the non-suspended material data archive in FIG. 12 and FIG. 13, respectively, the present invention is not limited thereto. For example, as needed or requested, the functions (e.g., performing clustering, etc.) of the suspended material data archive and the non-suspended material data archive may be exchanged (or performed in reverse), and the water quality management device 100 (or the main server device 200) may use both the operations of FIG. 12 and FIG. 13.

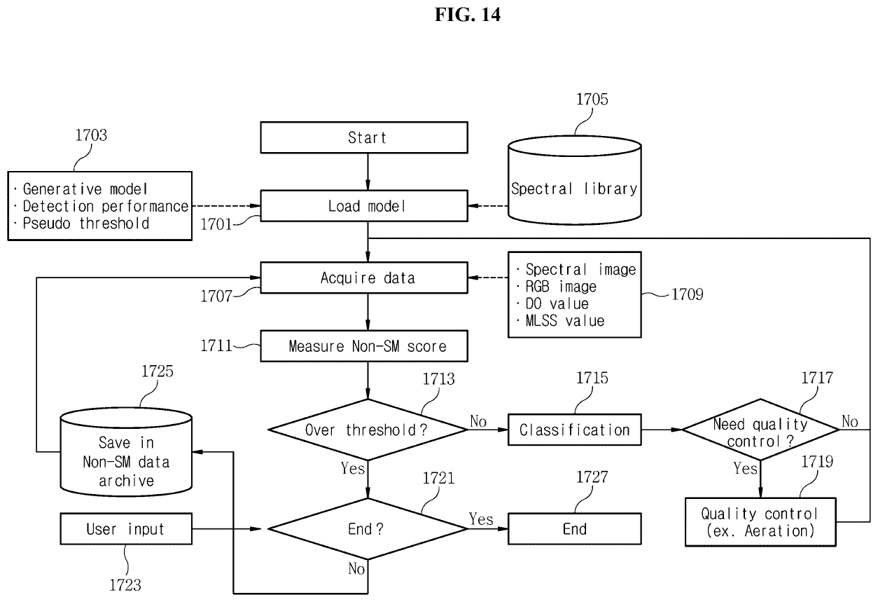

FIG. 14 is a diagram showing an example of a water quality management method according to the second embodiment of the present invention. Although the method in FIG. 14 is described based on the operation of the processor of the water quality management device, the present invention is not limited thereto, and the method in FIG. 14 may correspond to at least a part of the operation of the server processor of the main server device described in FIG. 10.

Referring to FIG. 14, in relation to the water quality management method according to the second embodiment of the present invention, in step 1701, the processor 150 of the water quality management device 100 may perform model loading. In relation to this, the water quality management device 100 may prepare a generative model, detection performance, and pseudo threshold for a plurality of spectral images in step 1703 and may prepare a spectral library (hyperspectral library) in step 1705. The steps 1703 and 1705 may be performed prior to the step 1701.

In step 1707, the processor 150 may perform data acquisition. In this regard, the water quality management device 100 may prepare in step 1709 at least some of the spectral data (hyperspectral data), RGB image, and sensing information (DO value, MLSS value) at a time (e.g., current time) at which water quality is to be checked. Meanwhile, the step 1709 may be performed prior to the steps 1701 and 1707 described above.

In step 1711, the processor 150 may measure a non-suspended material (non-SM) score. In relation to the score measurement, the water quality management device 100 may store and manage a score table according to a predefined threshold value for various spectral images (or including at least some of RGB images and sensing information). The processor 150 may calculate a non-SM score for a spectral image of the current time, and check in step 1713 whether the calculated non-SM score exceeds the predefined threshold value. If the non-SM score exceeds the predefined threshold value, classification may be performed on the spectral image of the current time in step 1715. In relation to the classification, the water quality management device 100 may store and manage a defined purification table in which the times and intensities of purification works are classified according to the degree of including suspended materials. For example, the water quality management device 100 may determine that water purification is necessary when the non-SM score exceeds the threshold value, and may determine that water purification is not necessary when the non-SM score does not exceed the threshold value.

Next, in step 1717, the processor 150 may check whether the current classification value needs water quality control. Upon determining that water quality control is not needed, the processor 150 may return to step 1707 and re-perform the subsequent operations. On the other hand, if water quality control is needed, the processor 150 may perform water quality control (e.g., aeration) in step 1719. For example, the processor 150 may transmit a warning message to a water purification device capable of performing the water purification operation on the stored water 50 or to the administrator terminal 300 that manages the water purification device. The warning message may include, for example, the classification value, and water purification information (e.g., at least some of the type and amount of chemicals used, the operating time and intensity of the water purification device) corresponding to the classification value may be provided to the administrator terminal 300 (or the water purification device).

Meanwhile, if the non-SM score is greater than or equal to the threshold value in step 1713, the processor 150 may check in step 1721 whether an event related to the end of processing occurs. In this regard, the processor 150 may perform step 1723, which supports a user input (user interaction) related to the end of processing. For example, the processor 150 may output a screen interface for a user input to the display 170, or perform interfacing (e.g., performing input transmission and reception operations) to receive a user input from the administrator terminal 300 that has requested water quality control of the current time. If there is no additional processing request for a predefined period of time or there is a user input requesting the end of processing, the processor 150 may terminate the routine on the water quality control in step 1727.

If there is no end event related to water quality control or if any additional input for water quality control is received, the processor 150 may record the currently processed data in a non-SM data archive in step 1725 and return to step 1707 to re-perform the subsequent operations.

### Third Embodiment

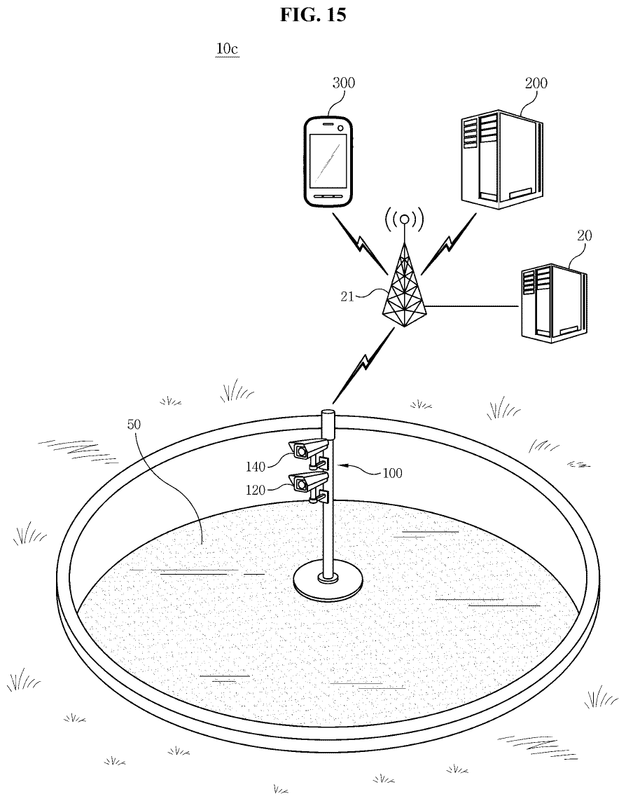

FIG. 15 is a diagram showing an example of a water quality management system according to the third embodiment of the present invention.

Referring to FIG. 15, the water quality management system 10c of the present invention may include stored water 50 (e.g., water stored in a tank), a water quality management device 100, a base station 21, a relay server device 20, a main server device 200, and an administrator terminal 300. Here, it is exemplified that the water quality management system 10b includes the base station 21 for communication between the water quality management device 100 and the relay server device 20, the main server device 200, or the administrator terminal 300, but the present invention is not limited thereto. For example, without the base station 21, the water quality management device 100 and the main server device 200 or the water quality management device 100 and the relay server device 20 may also be directly connected via a wired cable or a short-range wireless communication scheme. In this case, the base station 21 may be omitted from the water quality management system 10c. In addition, although it is exemplified that the relay server device 20 is included in the water quality management system 10c, the present invention is not limited thereto. For example, the relay server device 20 may be omitted or provided as being integrated with the main server device 200.

The stored water 50 may be water stored in a tank having a certain size. The tank that stores water may be at least one of various tank types such as a sewage tank of a sewage treatment plant or a water supply tank for water supply treatment. Therefore, the stored water 50 may be water stored in a sewage tank, water stored in a water supply tank, or the like. The stored water 50 in the present invention may include a suspended material, for example, depending on various external or internal conditions of the water tank. The stored water 50 including a suspended material may be purified through a purification process. The stored water 50 may be located within a shooting angle at which a spectral camera 120 and an RGB camera 140 disposed in the water quality management device 100 can photograph. In addition, the stored water 50 may be positioned so that it can be sensed by at least one sensor disposed in the water quality management device 100 or independently disposed at a certain place of the water tank.

The water quality management device 100 may include the spectral camera 120 that collects spectral images of the stored water 50, the RGB camera 140 that collects RGB images of the stored water 50, and a mounting structure that mounts the spectral camera 120 and the RGB camera 140. Although it is described that the water quality management device 100 includes both the spectral camera 120 and the RGB camera 140, the present invention is not limited thereto. For example, the water quality management device 100 may include only the spectral camera 120. Additionally, the water quality management device 100 according to the third embodiment of the present invention may further include at least one sensor that can collect sensing information related to the water quality of the stored water 50 in addition to the spectral images. The water quality management device 100 may detect the water quality of the stored water 50 based on at least some of the spectral image collected by the spectral camera 120, the RGB image collected by the RGB camera 140, and the sensing information collected by the at least one sensor. In this regard, the water quality management device 100 trains a model related to water quality measurement based on collected data (e.g., the spectral images collected by the spectral camera 120, at least some of the RGB image and sensing information collected together at the time of collecting the spectral images). In addition, the water quality management device 100 may construct a spectral library on whether there is a suspended material in the stored water 50, based on the collected spectral data, and determine whether to perform water quality management using the spectral library. In addition, the water quality management device 100 may classify the spectral data and, based on the classification result, determine whether to perform water quality management. The water quality management device 100 may determine whether to perform water quality management, based on at least one of the spectral library and the classification result. In this regard, the water quality management system 10c may further include a purification device for water quality management, and the water quality management device 100 may create a message according to whether water quality management is necessary, and provide the created message to the purification device.

Meanwhile, the water quality management device 100 may be configured to transmit the collected data (e.g., at least some of the spectral image collected by the spectral camera 120, the RGB image collected by the RGB camera 140, and the sensing information collected by the at least one sensor) related to the stored water 50 to the main server device 200. In this case, the water quality management device 100 may form a communication channel with (or may be directly connected to) the main server device 200 through the base station 21. The water quality management device 100 may transmit the collected data (e.g., at least some of the spectral image collected by the spectral camera 120, the RGB image collected by the RGB camera 140, and the sensing information collected by the at least one sensor) to the main server device 200.

The base station 21 may be spaced apart from the water quality management device 100 by a certain distance and disposed geographically at a distance or location where a wireless communication channel can be formed with the water quality management device 100. Alternatively, the base station 21 may be connected to the water quality management device 100 by wire. Additionally, the base station 21 may support forming at least one of communication channels between the water quality management device 100 and the main server device 200, between the water quality management device 100 and the relay server device 20, between the relay server device 20 and the main server device 200, between the water quality management device 100 and the administrator terminal 300, and between the main server device 200 and the administrator terminal 300.

The main server device 200 may form a communication channel with the water quality management device 100 through the base station 21 (or directly without the base station 21). The main server device 200 may receive the collected data (e.g., at least some of the spectral image collected by the spectral camera 120, the RGB image collected by the RGB camera 140, and the sensing information collected by the at least one sensor) from the water quality management device 100 and perform the learning of a water quality detection model based on the received data. Thereafter, the main server device 200 may perform spectral image analysis on the stored water 50 at a specific time based on the learning-completed water quality detection model, thereby estimating the water quality of the stored water 50. For example, based on at least some of the collected data, the main server device 200 may construct a spectral library in which data is scored based on whether the stored water 50 contains a suspended material (or at least one of the size of the suspended material, the extent (density) of the suspended material, and whether a water purification work is required due to the suspended material), and then perform water quality detection and water quality control using the constructed spectral library. Alternatively, the main server device 200 may perform classification on the collected data and, based on the classification result, determine whether to perform water quality management. Here, for the classification, the main server device 200 may perform clustering on the spectral data, perform spectrum comparison between the clustering result and a predefined cluster that requires water quality management, and determine whether to perform water quality control. The main server device 200 may transmit the water quality result (or water quality estimation result) of the stored water 50 to a designated administrator terminal 300 (or user terminal) or output it through a connected display device.

Meanwhile, if the learning of the water quality detection model and the estimation of the water quality result of the stored water 50 are designed to be processed in the water quality management device 100, the main server device 200 may be omitted. In this case, the water quality management device 100 may directly produce the water quality result and transmit it to the administrator terminal 300 or output it to a connected display.

Although it is described above that at least one sensor is included in the water quality management device 100, the present invention is not limited thereto. For example, the at least one sensor may be placed at a specific location of the stored water 50 without being included in the water quality management device 100, have a communication module on its own, and be connected to the relay server device 20 via the base station 21. Here, the communication module included in the at least one sensor may be connected to the relay server device 20 via a wired cable, or may be formed as a wireless communication module and connected to the relay server device 20 via the base station 21. The at least one sensor may collect sensing information on the stored water 50 according to predefined schedule information, and transmit the collected sensing information to the relay server device 20.

The relay server device 20 may be connected to at least one sensor, and transmit sensing information collected from the at least one sensor to at least one of the water quality management device 100 and the main server device 200 at regular intervals or in real time. Alternatively, the relay server device 20 may receive control information regarding at least one sensor from at least one of the water quality management device 100 and the main server device 200, and control the at least one sensor based on the received control information to collect sensing information. Meanwhile, as mentioned above, the relay server device 20 may be integrated with the main server device 200 or, if at least one sensor is included in the water quality management device 100, may be omitted.

The administrator terminal 300 may form a communication channel with at least one of the water quality management device 100, the relay server device 20, and the main server device 200 through the base station 21. The administrator terminal 300 may receive the estimated water quality result from at least one device. The administrator terminal 300 may output the water quality result through an output device (e.g., a display or an audio device). In another example, the administrator terminal 300 may receive a water purification operation request message according to the water quality result from at least one device. The administrator terminal 300 may output the received water purification operation request message. Alternatively, the administrator terminal 300 may output the water quality result and the water purification operation request message simultaneously to provide information so that the administrator can determine when to perform the water purification operation on the stored water 50. In an example, the administrator terminal 300 may be a terminal of an administrator who manages a water purification device capable of performing the water purification operation on the stored water 50. In this regard, the water quality management device 100 may further include a water purification device capable of performing water purification on the stored water 50, and the administrator terminal 300 may include the water purification device.

As described above, the water quality management system 10c according to the third embodiment of the present invention can acquire spectral images of the stored water 50, determine whether to control water quality management based on at least one of the spectral library that classifies data based on the relevance of a suspended material in the acquired spectral images and data classification values according to clustering of the spectral images, and control the message transmission to the purification device or the operation of the purification device based on the determination. Through this, the water quality management system 10c of the present invention can provide the advantages of more accurately measuring the water quality, thereby determining whether to manage water quality, and performing water quality management at various time points as needed.

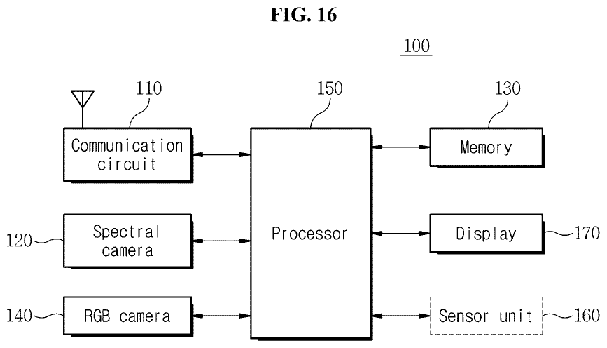

FIG. 16 is a diagram showing an example of components of a water quality management device according to the third embodiment of the present invention. In the following description, a form in which the relay server device 20 is integrated with the main server device 200 is exemplified. However, the present invention is not limited thereto, and the relay server device 20 may be separately included in the water quality management system 10c for the operation of a sensor unit 160 including at least one sensor.

Referring to FIG. 16, the water quality management device 100 according to the third embodiment of the present invention may include a communication circuit 110, a spectral camera 120, a memory 130, an RGB camera 140, a sensor unit 160, a display 170, and a processor 150. Here, if the water quality management device 100 is configured to perform a water quality management by using only spectral images, at least one of the RGB camera 140 and the sensor unit 160 may be omitted. In addition, the water quality management device 100 may further include a mounting structure for mounting the spectral camera 120 so that the spectral camera 120 can be oriented to an angle capable of capturing at least a portion of the stored water 50. In addition, the water quality management device 100 may further include a power supply (e.g., a permanent power supply or a battery) required for the operation of at least one of the above-described components, for example, the communication circuit 110, the spectral camera 120, the memory 130, the RGB camera 140, the sensor unit 160, the display 170, and the processor 150. Meanwhile, at least one of the components of the water quality management device 100 may be omitted.

The communication circuit 110 may form a communication channel with the main server device 200 (or the relay server device 20 or the administrator terminal 300) via the base station 21. Alternatively, the communication circuit 110 may form a communication channel directly with the main server device 200 or the relay server device 20 without going through the base station 21. If the main server device 200 is designed to perform the water quality management according to the third embodiment of the present invention, the communication circuit 110 may transmit spectral images collected by the spectral camera 120 (additionally at least some of RGB images and sensing information) to the main server device 200. On the other hand, a water quality management function may be performed independently by the water quality management device 100. In this case, the communication circuit 110 may transmit a warning message (e.g., a message requesting water purification work for the stored water 50) based on the water quality result to the main server device 200. Alternatively, the communication circuit 110 may transmit at least some of the water quality result and the warning message to a resident of a certain area or an administrator of a certain institution (e.g., the administrator terminal 300) under the control of the processor 150. The communication circuit 110 may also receive an artificial neural network algorithm required for fluid path diffusion prediction from the main server device 200. In relation to the above-described function support, the communication circuit 110 may include a plurality of communication modules.

The spectral camera 120 may be disposed to capture a spectral image for at least a section (or at least a point) of the stored water 50. The spectral camera 120 may capture the spectral image of the stored water 50 under the control of the processor 150. For example, when a predefined scheduled event occurs, the spectral camera 120 may capture a plurality of spectral images related to the stored water 50 in response to the occurrence of the event. For example, under the control of the processor 150 (or in response to a request of the main server device 200), the spectral camera 120 may acquire the plurality of spectral images related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). Alternatively, the spectral camera 120 may capture the plurality of spectral images related to the stored water 50 according to a predefined regular cycle. Alternatively, the spectral camera 120 may collect spectral images for constructing a spectral library or detecting various classification values.

The memory 130 may store at least one program or data required for the operation of the water quality management device 100. For example, the memory 130 may store at least some of a control program required for operating the spectral camera 120, a plurality of spectral images acquired by the spectral camera 120, a water quality detection model generated based on the plurality of spectral images, a spectral library that classifies data based on a suspended material relevance of the plurality of spectral images, classification values of data classified based on the clustering results of the plurality of spectral images, a spectral image acquired at the current time and a water quality result derived therefrom, a warning or notifying message corresponding to the water quality result. In addition, the memory 130 may store an artificial neural network algorithm or a machine learning algorithm required for the water quality detection model.

The RGB camera 140 may be disposed to have the same shooting angle or face the same shooting point as the shooting angle or shooting point of the spectral camera 120. The RGB camera 140 may acquire at least one RGB image of the stored water 50 while being synchronized with the time at which the spectral camera 120 captures a spectral image. For example, the RGB camera 140 may acquire at least one RGB image related to the stored water 50 in at least one of a case where new water flows into the tank, a case where at least a portion of the water stored in the tank is discharged, a case where the flow of the stored water 50 is changed, a case where the weather around the tank containing the stored water 50 changes (e.g., when it rains or snows), a case where the ambient temperature of the tank containing the stored water 50 changes, or a case where the ambient illuminance of the tank containing the stored water 50 changes (e.g., when the illuminance changes due to the formation of rain clouds). The acquired RGB image may be temporarily stored in the memory 130 and, in response to the control of the processor 150, may be transmitted to a designated device (e.g., the main server device 200). For example, the acquired RGB image may be output through the display 170 of the water quality management device 100.

The sensor unit 160 may be disposed in the water quality management device 100 and collect sensor information related to the water quality of the stored water 50 at the time when the spectral camera 120 acquires the spectral image. For example, the sensor unit 160 may include at least one of a dissolved oxygen (DO) sensor, a mixed liquor suspended solids (MLSS) sensor, a biochemical oxygen demand (BOD) sensor, and a chemical oxygen demand (COD) sensor, which can directly measure water quality information. In addition, the sensing information collected by the sensor unit 160 may be temporarily stored in the memory 130 and, depending on the design form, may be transmitted to the main server device 200. Meanwhile, at least one sensor included in the sensor unit 160 may be connected to the relay server device 20 and configured to collect sensing information in response to the control of the relay server device 20 and then transmit the sensing information to the water quality management device 100 or the main server device 200. The at least one sensing information collected by the sensor unit 160 may be used for at least one operation of water quality detection model generation and water quality result estimation. The sensor unit 160 may be configured to collect the sensing information at the time of spectral image acquisition. Alternatively, the sensor unit 160 may be configured to collect the sensing information at a predefined time or cycle or in real time.

The display 170 may output various information screens required for the operation of the water quality management device 100. For example, the display 170 may output a screen related to the operation of at least one of the spectral camera 120 and the RGB camera 140 of the water quality management device 100. The display 170 may output at least in part the operating information of the sensor unit 160 and the sensing information collected by the sensor unit 160. In addition, the display 170 may output a screen related to learning of a generative model (or a water quality detection model). The display 170 may output at least one of a screen related to constructing or operating a spectral library, a screen related to configuring classification values for a plurality of spectral images, and a screen related to operating the classification values. The display 170 may include a touch screen that supports a user input function. The display 170 may output at least one of the water quality result of the currently collected spectral image and the warning message corresponding to the water quality result under the control of the processor 150.

The processor 150 may control the spectral camera 120 to acquire a plurality of spectral images or a current spectral image related to the stored water 50 in response to a predefined event or a request from the main server device 200 or the administrator terminal 300. The processor 150 may perform at least one of an operation of constructing a spectral library to be used for water quality detection based on the plurality of acquired spectral images, an operation of creating classification values, and an operation of estimating the water quality result of the currently collected spectral image.

For example, the processor 150 may generate a water quality detection model based on a suspended material. The processor 150 may collect spectral images of the stored water 50 by using the spectral camera 120 and may generate a model capable of determining the presence or absence of a suspended material (SM) by analyzing the collected spectral images. For example, in the process of analyzing the acquired spectral image, the processor 150 may determine that a suspended material exists in the spectral image if the wavelength is relatively short compared to predefined criteria and the fluctuation range of reflectance is less than the predefined criteria when observing the situation. The processor 150 may determine that a suspended material does not exist in the spectral image if the wavelength is relatively long compared to the predefined criteria, the overall reflectance is high, and the variation range is greater than the predefined criteria. The predefined criteria may be the average value of the acquired spectral images or may be defined by the setting value of the administrator involved in model generation. For example, the consistency of the spectral images in the case where a suspended material exists may be higher than the consistency of the spectral images in the case where a suspended material does not exist. The processor 150 may use the high-consistent spectral images with suspended materials for learning of a generative model.

In relation to the generative model learning (or water quality detection model learning), the processor 150 may use the Auto-Encoder or the Generative Adversarial Network among artificial neural networks for deep learning. The processor 150 may use a traditional machine learning model in addition to a deep learning algorithm, and the present invention is not limited to deep learning and machine learning models. The processor 150 may output a user UI (e.g., a screen displaying a list of training data, a screen for selecting at least one item included in the list) through the display 170 so that a user (or an administrator involved in generative model learning) can manually designate a list of training data. The processor 150 may process label designation for each data sample in response to a user input. In the labeling operation, the processor 150 may include spectral data (or spectral image) in the label, and may also output at least one type of sensing information collected by the sensor unit 160 to the display 170 so that the user can refer to it during the labeling process.

In another example, the processor 150 may support semi-automatic designation of a label by a criterion allowed by the user. In applying the criterion, the processor 150 may include a spectral image (or spectral data) in the corresponding labeled data and output at least part of the sensing information collected by the sensor unit 160 to the display 170 for reference. In relation to the semi-automatic designation, the processor 150 may output to the display 170 a screen that allows the user to designate a specific criterion value (e.g., values obtained by multiplying the average value and the standard deviation of frequency values of spectral images by a predefined coefficient value) for determining the presence or not of a suspended material, and determine the specific criterion value for determining the presence or not of a suspended material in the spectral image by a user input. When the criterion value is determined, the processor 150 may analyze the spectrum of spectral images to check whether frequency distribution is less than or greater than the criterion value, and thereby automatically determine the presence or not of a suspended material.

The processor 150 may construct a spectral library based on the suspended material relevance of spectral images. For example, the processor 150 may perform clustering on spectral images and construct a suspended material data archive or a non-suspended material data archive based on the cluster analysis produced as a result of the performance. In this process, the processor 150 may calculate a score for the spectral image based on the degree of including a suspended material or the size of a suspended material, and determine whether water quality management is necessary for the corresponding spectral image based on the calculated score. In addition, the processor 150 may pre-store an image classification table (or classification values) indicating a need for water quality management for respective spectral images, and compare an image classification value for a currently acquired spectral image with the pre-stored classification values to determine whether water quality management is needed. In relation to creating the classification table (or classification criteria), the processor 150 may perform convolutional neural network (CNN) learning, which is a deep learning technique, and may extract an optimal feature map to perform pixel classification on the spectral image.

The processor 150 may determine whether there is a suspended material in the currently acquired spectral image, by operating at least one of the learned generative model, the spectral library, and the image classification in parallel. If a restoration error is greater than a predefined threshold value, the processor 150 may consider or determine this to be a situation that has not been observed during the learning process and in which no suspended material exists. If the restoration error is less than the predefined threshold value, the processor 150 may consider this as a situation that has been observed (or observed similarly) during the learning process and determine that a suspended material exists. In an example, the determination criterion may use a threshold at a point where the classification performance index (e.g., F1-score) is maximum during the learning process. Here, the threshold may be adjusted or designated by a user input.

In relation to a semi-automatic spectral library construction through a suspended material discrimination model, if the processor 150 determines that a suspended material exists in the spectral image according to the suspended material discrimination method described above, the processor 150 may store the spectral image determined to have a suspended material in a suspended material data archive area (or a separate suspended material archive) of the memory 130. If the processor 150 determines that a suspended material does not exist, the processor 150 may store the spectral image in the non-suspended material (non-SM) data archive area (or non-suspended material data archive) of the memory 130. If the number of data held by the suspended material archive exceeds a predefined certain amount or a user request occurs, the processor 150 may cluster the data stored in the archive by similar characteristics. An example of a clustering method may be K-means clustering, but the present invention is not limited thereto. Based on settings or user input, the processor 150 may perform the same clustering process on data stored in the non-suspended material (non-SM) data archive. When clustering is completed, the processor 150 may output the clustering result to the display 170 to allow the user (or administrator) to check the clustering result, and may confirm the information indicated by the cluster in response to a user input. In this process, the information output to the display 170 may include the result of a specific cluster, a spectral image corresponding to the cluster, and/or sensing information collected together when the spectral image is collected. When the user confirms the cluster information, the processor 150 may add the confirmed cluster to the spectral library. For example, the confirmed information may include one or more of information necessary for water quality management, such as water quality status, types of suspended materials, and measures to be taken for water quality management.

Based on setting or a user request, the processor 150 may support determining information by referring to other data existing in a hyperspectral library. In an example, based on setting or a user input, the processor 150 may delete or overwrite information existing in the hyperspectral library. Based on setting or a user request, the processor 150 may support clustering of at least one of the suspended material data archive and the non-suspended material data archive and a clustering-based water quality management function.

As an indicator of water quality status in relation to water quality management, the processor 150 may use at least one of a type of using the presence or not of a suspended material, a type of using a water quality status indicator utilizing a spectral library, and a type of using image classification for spectral images. The water quality management device 100 may selectively use at least one of the above-described various types, and may use at least one of the indication of the presence or not of a suspended material, the water quality status indication based on a spectral library, and the indication of image classification values as needed during operation. In an example, the processor 150 may first determine the presence or not of a suspended material in the stored water 50, and if a suspended material is present, may determine the current detailed status by utilizing at least one of the spectral library and the image classification values.

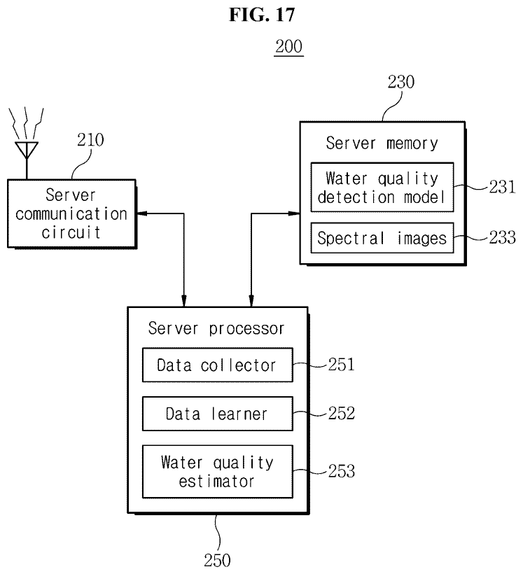

FIG. 17 is a diagram showing an example of components of a main server device according to the third embodiment of the present invention. As described above, if the water quality management function of the present invention is designed to be performed solely by the water quality management device 100, the main server device 200 may be omitted.

Referring to FIG. 17, the main server device 200 of the present invention may include a server communication circuit 210, a server memory 230, and a server processor 250.

The server communication circuit 210 may form a communication channel with the water quality management device 100 through the base station 21 or directly. The server communication circuit 210 may collect a plurality of spectral images and a current spectral image from the water quality management device 100 at a designated cycle or in response to the occurrence of a predefined event. The server communication circuit 210 may transmit a water quality result and a corresponding warning message to a designated administrator terminal 300 or institution.

The server memory 230 may store at least one program or data required for the operation of the main server device 200. For example, the server memory 230 may store a water quality detection model 231. Additionally, the server memory 230 may store a plurality of spectral images 233 (or spectral data) received from the water quality management device 100. Also, the server memory 230 may store at least one of a spectral library and image classification values. Also, the server memory 230 may store at least one sensing information received from the water quality management device 100. For example, the server memory 230 may store the plurality of spectral images 233 received from the water quality management device 100 and used to generate the water quality detection model 231. The server memory 230 may store the water quality detection model 231 generated based on the plurality of spectral images 233. In addition, the server memory 230 may also store a current spectral image used for a current water quality check.

The server processor 250 may control the transmission and processing of signals required for the operation of the main server device 200, the storage of results, the transmission of results, or the transmission of messages corresponding to results. In this regard, the server processor 250 may include a data collector 251, a data learner 252, and a water quality estimator 253.

The data collector 251 may request a plurality of spectral images related to the stored water 50 from the water quality management device 100 at a predefined cycle or in response to the occurrence of a predefined event. According to the third embodiment, the data collector 251 may collect a plurality of spectral images related to the stored water 50 for a certain period of time prior to a time for the generation of a generative model (or a water quality detection model) to be used for water quality management. In another example, the data collector 251 may request at least some of sensing information and RGB images collected together at the time of collecting the spectral images from the water quality management device 100 and store them in the server memory 230.

The data learner 252 may perform learning on a plurality of spectral images collected by the data collector 251. In this regard, the data learner 252 may perform learning on the plurality of spectral images related to the stored water 50 based on a learning algorithm stored in the server memory 230, thereby generating a water quality detection model. In this process, the data learner 252 may perform at least a part of the operation of the processor 150 described above in FIG. 16. For example, the data learner 252 may classify the spectral images 233 stored in the server memory 230 into spectral images from which a suspended material is detected and spectral images from which a suspended material is not detected, and perform learning on each of the classified spectral images. For example, the data learner 252 may perform at least one of learning on a generative model including a suspended material, calculation of scores based on a suspended material relevance, and learning on a generative model including no suspended material. The data learner 252 may perform clustering on data classified according to the presence or absence of a suspended material, and store a cluster confirmed based on a clustering result (or a cluster confirmed based on a user input) and corresponding spectral images (or additionally, sensing information collected at the time of collecting the spectral images) in a spectral library.

Upon completing learning on the plurality of spectral images 233 and generating the water quality detection model 231, the data learner 252 may provide a guidance message to the administrator terminal 300 (or the water quality management device 100) that requested it. The data learner 252 may store the generated water quality detection model 231 in the server memory 230. For example, the data learner 252 may perform labeling of the plurality of spectral images 233, classification of the plurality of labeled spectral images 233 (e.g., classification into a suspended material data set (or a suspended material spectral library) including spectral images with a suspended material and a non-suspended material data set (or a non-suspended material spectral library) without a suspended material), and classification into training and validation (e.g., classification into a training data set of a certain ratio (e.g., 80%, changeable) for training the suspended material data set and a validation data set of a certain ratio (e.g., 20%, changeable) for validation), generate the water quality detection model 231 by using only the training data set, and then perform a performance check of the water quality detection model 231 by analyzing a restoration error after inputting the validation data set into the water quality detection model 231.

In relation to labeling, the data learner 252 may assign indexes to a plurality of spectral images and separate labeled spectral images and unlabeled spectral images based on the indexes. The data learner 252 may perform an integrity check (e.g., outputting at least some of sensing information, RGB images, and spectral images to the display 170 so that the user can check them, and performing an integrity check in response to a user input) on the unlabeled spectral images. The data learner 252 may perform a suspended material check (e.g., outputting at least some of sensing information, RGB images, and spectral images to the display 170 so that the user can check them, and determining whether or not there is a suspended material in response to a user input) on the spectral images that have passed the integrity check. In another example, the data learner 252 may perform image classification on spectral images by using deep learning, determine the degree of including a suspended material regarding image classification values, determine whether water quality management is needed (or water quality control information) based on the degree of including a suspended material, and store/manage them in the server memory 230. Here, in relation to matching between a water quality estimation value and control information based on water quality estimation for the image classification values, the data learner 252 may output related information to the display 170 and, based on a user input, determine the water quality estimation value. Alternatively, the data learner 252 may perform matching of water quality estimation values for current spectral images by using a table of water quality estimation values by predefined image classification values.

The water quality estimator 253 may calculate the restoration error of the currently collected spectral image by using the water quality detection model 231 (or the generative model), and estimate the water quality result of the currently collected spectral image based on the calculated restoration error. Alternatively, the water quality estimator 253 may estimate the water quality result of the currently collected spectral image by using a spectral library that stores the clustering result of spectral images. For example, the water quality estimator 253 may analyze the spectrum of the currently collected spectral image, perform clustering on the spectrum, compare the clustering result with clusters pre-stored in the spectral library (e.g., a cluster determined as a spectral image including a suspended material, a cluster determined as a spectral image including no suspended material, or clusters classified into various water quality states), and estimate the water quality result of the currently collected spectral image based on the comparison result. In addition, the water quality estimator 253 may perform image classification by performing deep learning on spectral images, perform water quality estimation by comparing an image classification value with pre-stored image classification values, and collect water quality control information according to the water quality estimation. The water quality estimator 253 may transmit the water quality result estimation value to the administrator terminal 300 or transmit a guidance message or warning message according to the water quality result to the administrator terminal 300.

Meanwhile, the operations of the data collector 251, the data learner 252, and the water quality estimator 253 described in the server processor 250 may be configured in the processor 150 of the water quality management device 100 described in FIG. 16, and the water quality management device 100 may be configured to generate a water quality detection model, detect water quality by using the generated water quality detection model, and perform operations according to the detected water quality results. Therefore, among the operations related to the generation of the water quality detection model 231 and the water quality check in FIG. 16, parts that are not described in FIG. 17 may be supplemented and understood as described in FIG. 17, and similarly, parts of the description of FIG. 17 that are not described in FIG. 16 may be supplemented and understood as described in FIG. 16.

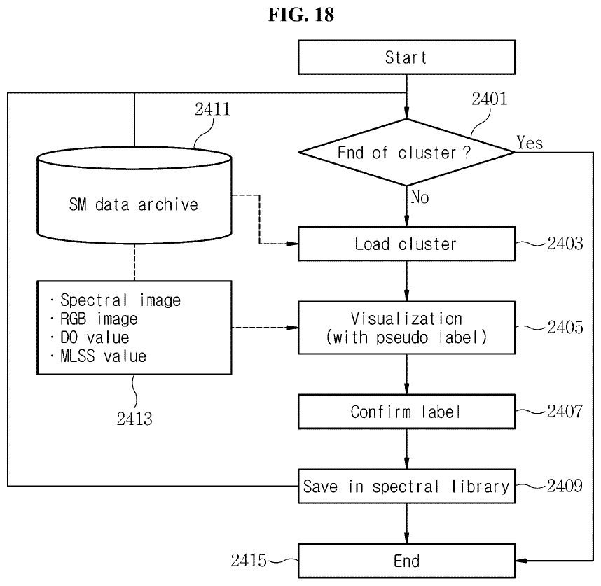

FIG. 18 is a diagram showing an example of a spectral library preparation method according to the third embodiment of the present invention. Although the method in FIG. 18 is described based on the operation of the processor of the water quality management device, the present invention is not limited thereto, and the method in FIG. 18 may correspond to at least a part of the operation of the server processor of the main server device described in FIG. 17.

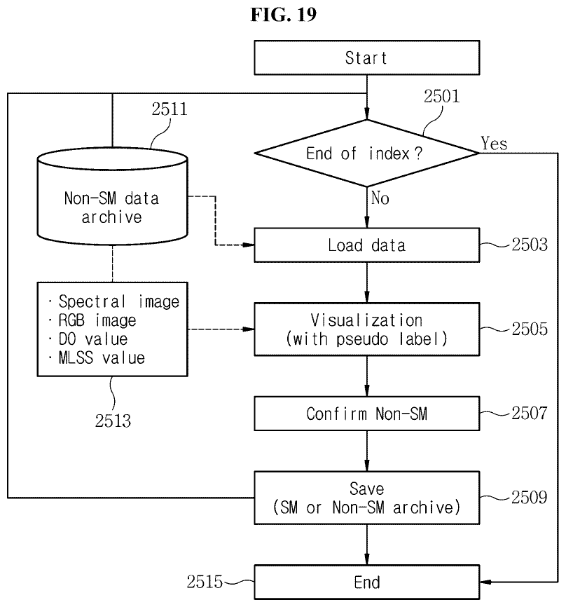

Referring to FIG. 18, in a method for operating the water quality management device related to the spectral library preparation according to the second embodiment of the present invention, the processor 150 of the water quality management device 100 may check whether a cluster to be currently processed is the last cluster (end of cluster?) in step 2401. In this regard, the processor 150 may perform information processing capable of recognizing the last cluster for clusters corresponding to a plurality of spectral images. For example, the last cluster may have a predefined specific symbol or data differently from other clusters. If the cluster to be currently processed is not the last cluster, the processor 150 may load the cluster in step 2403. In this regard, the processor 150 may acquire suspended material data (or a spectral image, or cluster generated by performing clustering on a spectral image) from a suspended material data archive 2411. Meanwhile, the cluster order check in step 2401 and the cluster loading in step 2403 described above may be replaced with the index order check and data loading operations of FIG. 19 described below.

In step 2405, the processor 150 may perform visualization for the loaded cluster. In this operation, the processor 150 may output a pseudo label of the loaded cluster together to the display 170, and output at least some of the spectral data (hyperspectral data), RGB image, and sensing information (DO value, MLSS value) stored in a storage 2413 (or the memory 130) to the display 170. Through the visualization process, the processor 150 may output a clustering result (e.g., a graph) corresponding to the spectral image to the display 170.

In step 2407, the processor 150 may perform label confirmation. For example, if the processor 150 determines that the data output through visualization is data including a suspended material, the processor 150 may confirm the label. In step 2409, the processor 150 may store the labeled cluster in the spectral library (save in hyperspectral library) and then return to step 2401. In this operation, the processor 150 may update a spectral library area of the suspended material data archive.

If the current cluster is the last cluster in step 2401, the processor 150 may end the spectral library preparation operation. Here, information indicating the last cluster may not include actual cluster information separately, but may include a predefined specific symbol or data indicating the last cluster.

FIG. 19 is a diagram showing another example of a spectral library preparation method according to the third embodiment of the present invention. Although the method in FIG. 19 is described based on the operation of the processor of the water quality management device, the present invention is not limited thereto, and the method in FIG. 19 may correspond to at least a part of the operation of the server processor of the main server device described in FIG. 17.

Referring to FIG. 19, in relation to another example of a method for operating the water quality management device related to the spectral library preparation according to the third embodiment of the present invention, the processor 150 of the water quality management device 100 may check whether an index of information to be currently processed is the last index (end of index?) in step 2501. In this regard, the processor 150 may assign indexes to a plurality of spectral images and check whether the index corresponding to the spectral image to be currently processed is the last index. If the index to be currently processed is not the last index, the processor 150 may load data in step 2503. In this regard, the processor 150 may acquire and load non-suspended material data (or spectral image) from a non-suspended material data archive 2511. Meanwhile, index order checking in step 2501 and data loading in step 2503 described above may be replaced with the cluster order checking and cluster loading steps described in FIG. 18 above. That is, although FIG. 18 describes a method of using clustering in the process of preparing the SM data archive (or the SM spectral library), and FIG. 19 describes a method of using an index in the process of preparing the non-SM data archive (or the non-SM spectral library), the present invention is not limited thereto. For example, the process of preparing the SM data archive (or the SM spectral library) described in FIG. 18 above may be replaced with a process of preparing the non-SM data archive, and the processor 150 of the water quality management device 100 may operate a method of using clustering in the process. In addition, although FIG. 19 describes a method of using an index in the process of preparing the non-SM data archive (or the non-SM spectral library), the spectral library preparing operation in FIG. 19 may be replaced with an operation of preparing the SM data archive, and the processor 150 of the water quality management device 100 may prepare the spectral library based on an index order check in the operation.

In step 2505, the processor 150 may perform visualization for the loaded data (e.g., spectral image). In this operation, the processor 150 may output a pseudo label of the loaded data together to the display 170, and output at least some of the spectral data (hyperspectral data), RGB image, and sensing information (DO value, MLSS value) stored in a storage 2513 (or the memory 130) to the display 170. Through the visualization process, the processor 150 supports checking on the display 170 whether a non-suspended material corresponding to a spectral image is included.

In step 2507, the processor 150 may perform non-SM confirmation. For example, if the processor 150 determines that the data output through visualization is non-SM data including no suspended material, the processor 150 may confirm the currently processed data as non-SM data. Alternatively, if the processor 150 determines that the data output through visualization is SM data including a suspended material, the processor 150 may confirm the currently processed data as SM data. The SM data confirmation may be based on a user input.

In step 2509, the processor 150 may store the confirmed cluster in a designated archive (save SM or non-SM archive) and then return to step 2501. In this operation, the processor 150 may store the non-SM data in a non-SM data archive 2511. In step 2501, if the index to be currently processed is the last index, the processor 150 may end the spectral library preparation operation.

Meanwhile, although the spectral library generation method is described based on the suspended material data archive and the non-suspended material data archive in FIG. 18 and FIG. 19, respectively, the present invention is not limited thereto. For example, as needed or requested, the functions (e.g., performing clustering, etc.) of the suspended material data archive and the non-suspended material data archive may be exchanged (or performed in reverse), and the water quality management device 100 (or the main server device 200) may use both the operations of FIG. 18 and FIG. 19.

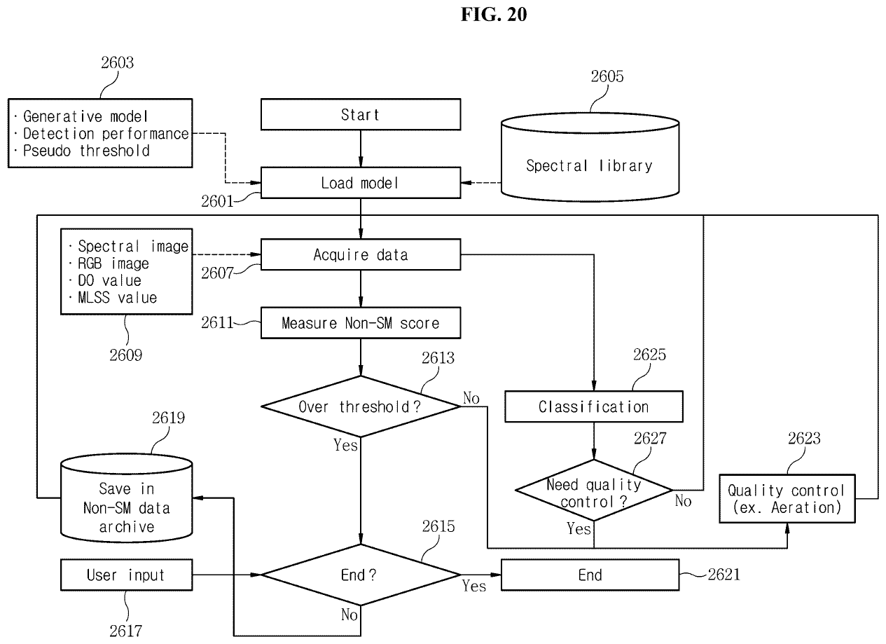

FIG. 20 is a diagram showing an example of a water quality management method according to the third embodiment of the present invention. Although the method in FIG. 20 is described based on the operation of the processor of the water quality management device, the present invention is not limited thereto, and the method in FIG. 20 may correspond to at least a part of the operation of the server processor of the main server device described in FIG. 17.

Referring to FIG. 20, in relation to the water quality management method according to the third embodiment of the present invention, in step 2601, the processor 150 of the water quality management device 100 may perform model loading. In relation to this, the water quality management device 100 may pre-store a generative model, detection performance, and pseudo threshold for a plurality of spectral images in step 2603 and may prepare a spectral library (hyperspectral library) in step 2605. The steps 2603 and 2605 may be performed prior to the step 2601. The spectral library may be at least one of the spectral libraries described above in FIG. 18 or FIG. 19.

In step 2607, the processor 150 may perform data acquisition. In this regard, the water quality management device 100 may prepare in step 2609 at least some of the spectral data (hyperspectral data), RGB image, and sensing information (DO value, MLSS value) at a time (e.g., current time) at which water quality is to be checked. Meanwhile, the step 2609 may be performed prior to the steps 2601 and 2607 described above. The processor 150 of the water quality management device 100 may control the spectral camera 120 to acquire spectral data, and operate the RGB camera 140 to acquire RGB images. Additionally, in order to acquire sensing information, the water quality management device 100 may collect relevant sensing information from at least one sensor.

After data acquisition, the processor 150 of the water quality management device 100 may operate a first water quality management method including steps 2611 to 2613 and a second water quality management method including steps 2625 to 2627 in parallel.

First, in relation to the first water quality management method, in step 2611, the processor 150 may measure a non-suspended material (non-SM) score. In relation to the score measurement, the water quality management device 100 may store and manage a score table according to a predefined threshold value for various spectral images (or including at least some of RGB images and sensing information). The processor 150 may calculate a non-SM score for a spectral image of the current time, and check in step 2613 whether the calculated non-SM score exceeds the predefined threshold value. If the non-SM score exceeds the predefined threshold value, the processor 150 may check in step 2615 whether the water quality management process is ended. In relation to whether the water quality management process is ended, the processor 150 may determine that there is no suspended material if the non-SM score is less than the predefined threshold value, and in this process, the processor 150 may receive a user input (user interaction) in step 2617. For example, the processor 150 may output the non-SM score to the display 170 and output a screen that allows the user to select a user input regarding whether to end the water quality management process. If there is no user input requesting the end of the water quality management process (or there is a user input requesting to continue the water quality management process) or the non-SM score is greater than or equal to the predefined threshold value, the processor 150 may update a non-SM data archive in step 2619, return to step 2607, and re-perform the subsequent operations.

On the other hand, if a user input requesting the end of the water quality management processing occurs or if the non-SM score is less than the predefined threshold value, the processor 150 may end the process in step 2621 and output a result (e.g., information indicating that water quality management processing has been ended) accordingly.

Although it is described that if the non-SM measurement score is greater than or equal to the threshold value in step 2613, step 2615 for checking whether to end the water quality management process is performed, the present invention is not limited thereto. For example, when the non-SM measurement score is greater than or equal to the threshold value in step 2613, the process may branch to step 2619 to store at least in part the non-SM measurement score and the spectral images (or related data) corresponding to the non-SM measurement score in the non-SM data archive, and then the subsequent step (e.g., 2607) may be performed again.

In relation to the second water quality management method, after step 2607, the processor 150 may perform classification on the spectral image of the current time. In relation to the classification, the water quality management device 100 may store and manage a defined purification table in which the times and intensities of purification works are classified according to the degree of including suspended materials. For example, the water quality management device 100 may determine that water purification is necessary when the non-SM score exceeds the threshold value, and may determine that water purification is not necessary when the non-SM score does not exceed the threshold value.

Next, in step 2617, the processor 150 may check whether the current classification value needs water quality control. Upon determining that water quality control is not needed, the processor 150 may return to step 2607 and re-perform the subsequent operations. On the other hand, if water quality control is needed, the processor 150 may perform water quality control (e.g., aeration) in step 2623. For example, the processor 150 may transmit a warning message to a water purification device capable of performing the water purification operation on the stored water 50 or to the administrator terminal 300 that manages the water purification device. The warning message may include, for example, the classification value, and water purification information (e.g., at least some of the type and amount of chemicals used, the operating time and intensity of the water purification device) corresponding to the classification value may be provided to the administrator terminal 300 (or the water purification device).

Meanwhile, if the non-SM score does not exceed the threshold value in step 2613 (over threshold?→No), the processor 150 may perform control related to water quality management in step 2623 described above. After step 2623, the processor 150 may return to step 2607 and re-perform the subsequent operations.

As described above, in the case of the water quality management control method according to the third embodiment of the present invention, while performing the operation of updating the non-suspended material data archive based on the non-suspended material score measurement and comparison with the threshold value, it is possible to perform control regarding water quality management based on at least one of the non-suspended material score, classification of the acquired data, and whether water quality management is necessary.

For example, the water quality management device of the present invention and the operating method thereof can determine the level of pollution by looking at the spectral library regardless of the presence or absence of suspended materials, and this function can provide related services to customers and users such as sewage treatment plants that require water resource management, and can provide such services to customers and users through a client system when a server is constructed. In addition, the present invention can provide a monitoring function by utilizing sensor data of the device, can provide a result of determining whether or not there is a suspended material through a suspended material detection model, and can provide an interface for the suspended material detection model to adjust a threshold value for the suspended material determination. In addition, the present invention can automate the construction of a spectral library (hyperspectral library) through the suspended material detection model, and can provide selective options of automatic, semi-automatic, and manual by allowing customers and users to provide additional information through the interface during the process of constructing the spectral library. In addition, the present invention can additionally provide a water quality status analysis result through the suspended material detection model and the spectral library to the monitoring function, and can display a water quality inspection necessity notification to a water quality management manager and a system user of a customer company, so that for situations such as the spectral library where there are no previously observed records, it is possible to recommend the operation of the water quality management system based on a probabilistic combination of similar situations.

While the description contains many specific implementation details, these should not be construed as limitations on the scope of the present invention or of what may be claimed, but rather as descriptions of features that may be specific to particular embodiments of particular invention.

Also, although the description describes that operations are performed in a predetermined order with reference to a drawing, it should not be construed that the operations are required to be performed sequentially or in the predetermined order, which is illustrated to obtain a preferable result, or that all of the illustrated operations are required to be performed. In some cases, multi-tasking and parallel processing may be advantageous. Also, it should not be construed that the division of various system components are required in all types of implementation. It should be understood that the described program components and systems are generally integrated as a single software product or packaged into a multiple-software product.

The description shows the best mode of the present invention and provides examples to illustrate the present invention and to enable a person skilled in the art to make and use the present invention. The present invention is not limited by the specific terms used herein. Based on the above-described embodiments, one of ordinary skill in the art can modify, alter, or change the embodiments without departing from the scope of the present invention.

Accordingly, the scope of the present invention should not be limited by the described embodiments and should be defined by the appended claims.

### REFERENCE NUMERALS

10a, 10b, 10c: Water quality management system
100: Water quality management device
110: Communication circuit
120: Spectral camera
130: Memory
140: RGB camera
150: Processor
160: Sensor unit
170: Display
200: Server device
210: Server communication circuit
230: Server memory
250: Server processor
300: Administrator terminal

What is claimed is:

1. A water quality management device comprising:
a spectral camera acquiring a plurality of spectral image related to stored water;
a memory; and
a processor functionally connected to the spectral camera and the memory, and configured to:
collect the plurality of spectral images for at least a portion of the stored water in response to a predefined event occurrence,
classify the plurality of spectral images into a suspended material data set and a non-suspended material data set based on a presence or absence of a suspended material in the spectral images,
perform learning to generate a water quality detection model used to detect water quality by using at least a portion of the suspended material data set and the non-suspended material data set, and
store the water quality detection model generated through the learning.

2. The device of claim 1, wherein the processor is configured to:
assign indexes to the plurality of spectral images acquired through the spectral camera, and
perform labeling on the plurality of spectral images for the assigned indexes.

3. The device of claim 2, wherein the processor is configured to:
check whether a label exists for a spectral image of a specific index among the assigned indexes, and if there is no label, perform an integrity check of the spectral image of the specific index,
check a presence or absence of a suspended material in the spectral image that has undergone the integrity check, and
perform labeling for the presence or absence of the suspended material.

4. The device of claim 3, wherein the processor is configured to:
perform the integrity check based on at least one of an RGB image, dissolved oxygen, and mixed liquor suspended solids (MLSS) collected together at a time when the spectral image of the specific index is collected in relation to the integrity check.

5. The device of claim 2, wherein the processor is configured to:
in relation to the suspended material check,
output at least one of an RGB image, dissolved oxygen, and mixed liquor suspended solids (MLSS) collected together at a time when the spectral image of the specific index among the assigned indexes is collected, and confirm the presence or absence of the suspended material in response to a user input.

6. The device of claim 1, wherein the processor is configured to:
divide the suspended material data set into a training data set and a validation data set with a predefined ratio,
generate the water quality detection model based on the training data set, and
perform a performance evaluation on the water quality detection model by using the validation data set.

7. An operating method of a water quality management device, the method comprising:
by a processor of the water quality management device, collecting a plurality of spectral images for at least a portion of stored water in response to a predefined event occurrence;
classifying the plurality of spectral images into a suspended material data set and a non-suspended material data set based on a presence or absence of a suspended material in the spectral images;
performing learning to generate a water quality detection model used to detect water quality by using at least a portion of the suspended material data set and the non-suspended material data set; and
storing the water quality detection model generated through the learning.

8. The method of claim 7, wherein classifying includes:
assigning indexes to the plurality of spectral images acquired through the spectral camera, and performing labeling on the plurality of spectral images for the assigned indexes,
wherein labeling includes:
checking whether a label exists for a spectral image of a specific index among the assigned indexes, and if there is no label, performing an integrity check of the spectral image of the specific index,
checking a presence or absence of a suspended material in the spectral image that has undergone the integrity check, and
performing labeling for the presence or absence of the suspended material, and
wherein checking the presence or absence of the suspended material includes:
outputting at least one of an RGB image, dissolved oxygen, and mixed liquor suspended solids (MLSS) collected together at a time when the spectral image of the specific index is collected, and confirming the presence or absence of the suspended material in response to a user input.

9. The method of claim 7, wherein classifying includes:
dividing the suspended material data set into a training data set and a validation data set with a predefined ratio,
generating the water quality detection model based on the training data set, and
performing a performance evaluation on the water quality detection model by using the validation data set.

10. A water quality management device comprising:
a spectral camera acquiring a plurality of spectral image related to stored water;
a memory storing a water quality detection model generated based on the plurality of spectral images; and
a processor functionally connected to the spectral camera and the memory, and configured to:
collect a spectral image for water quality management of the stored water at a current time,
calculate a non-suspended material score for the spectral image at the current time by using the water quality detection model,
perform classification for water quality management for the spectral image if the calculated non-suspended material score exceeds a predefined threshold value, and
control the water quality management based on a value of the classification.

11. The device of claim 10, wherein the memory comprises:
a spectral library including at least one of a suspended material data archive and a non-suspended material data archive, and
wherein the processor is configured to:
calculate the non-suspended material score based on at least one of the suspended material data archive or the non-suspended material data archive for the spectral image.

12. The device of claim 10, wherein the memory comprises:
a spectral library including a suspended material data archive, and
wherein the processor is configured to:
perform clustering on the spectral image at the current time, and calculate the non-suspended material score by comparing the cluster resulting from the clustering with clusters pre-stored in the suspended material data archive of the memory.

13. The device of claim 10, wherein the processor is configured to:
store information about the spectral image in a non-suspended material data archive of the memory if the non-suspended material score is less than the predefined threshold value.

14. The device of claim 10, wherein the processor is configured to:
in relation to performing the classification, define a purification intensity differently depending on a degree of a suspended material included in the spectral image, and produce a classification value of the current spectral image based on classification values pre-stored in the memory.

15. The device of claim 14, wherein the processor is configured to:
transmit a warning message including the purification intensity corresponding to the classification value to a water purification device capable of performing water purification on the stored water or to an administrator terminal of an administrator managing the water purification device.

16. A water quality management method comprising:
by a processor of a water quality management device, collecting a spectral image related to stored water at a current time;
calculating a non-suspended material score for the spectral image at the current time by using a water quality detection model generated based on a plurality of spectral images;
performing classification for water quality management for the spectral image if the calculated non-suspended material score exceeds a predefined threshold value; and
controlling the water quality management based on a value of the classification.

17. The method of claim 16, wherein calculating the non-suspended material score includes:
based on a spectral library including at least one of a suspended material data archive and a non-suspended material data archive, calculating the non-suspended material score based on at least one of the suspended material data archive or the non-suspended material data archive for the spectral image,
wherein performing the classification includes:
defining a purification intensity differently depending on a degree of a suspended material included in the spectral image, and producing a classification value of the current spectral image based on classification values pre-stored in memory, and
wherein controlling includes:
transmitting a warning message including the purification intensity corresponding to the classification value to a water purification device capable of performing water purification on the stored water or to an administrator terminal of an administrator managing the water purification device.

18. A water quality management device comprising:
a spectral camera acquiring a plurality of spectral image related to stored water;
a memory storing a spectral library that classifies data on a suspended material relevance of the plurality of spectral images and storing image classification values derived through deep learning for the plurality of spectral images; and
a processor functionally connected to the spectral camera and the memory, and configured to:
collect a current spectral image for water quality management of the stored water at a current time,
estimate the water quality of the current spectral image by using at least one of a process of identifying the suspended material relevance of the current spectral image by using the spectral library and a process of identifying an image classification value of the current spectral image by using the image classification values, and
based on a result of estimating the water quality, determine whether to perform purification processing for the stored water at the current time.

19. The device of claim 18, wherein the processor is configured to:
measure a non-suspended material score for the current spectral image by using the spectral library,
if the non-suspended material score exceeds a threshold value, generate control information for the purification processing for the stored water at the current time, and
transmit the control information to a purification device for the purification processing.

20. The device of claim 18, wherein the processor is configured to:
measure a non-suspended material score for the current spectral image by using the spectral library,
if the non-suspended material score exceeds a threshold value, output a screen for checking whether to end a process related to the water quality management, and
if there is no user input related to the end of the process, store the current spectral image in a non-suspended material data archive.

21. The device of claim 18, wherein the processor is configured to:
measure a non-suspended material score for the current spectral image by using the spectral library, and
if the non-suspended material score exceeds a threshold value, store the current spectral image in a non-suspended material data archive.

22. The device of claim 18, wherein the processor is configured to:
compare the image classification values with the image classification value for the current spectral image,
if the image classification value for the current spectral image has a value requiring purification processing as a result of comparison, generate control information for the purification processing, and
transmit the control information to a purification device for the purification processing.

23. An operating method of a water quality management device, the method comprising:
by a processor of the water quality management device, loading, from a memory, a spectral library that classifies data on a suspended material relevance of a plurality of spectral images, and image classification values derived through deep learning for the plurality of spectral images;
by the processor, collecting a current spectral image for water quality management of stored water at a current time by controlling a spectral camera;
estimating the water quality of the current spectral image by using at least one of a process of water quality estimation based on the suspended material relevance of the current spectral image using the spectral library and a process of water quality estimation based on an image classification value of the current spectral image calculated based on the image classification values; and
based on a result of estimating the water quality, determine whether to perform purification processing for the stored water at the current time.

24. The method of claim 23, wherein estimating includes:
measuring a non-suspended material score for the current spectral image by using the spectral library, and if the non-suspended material score exceeds a threshold value, determining the water quality of the stored water at the current time as requiring purification processing, or
comparing the image classification values with the image classification value for the current spectral image, and if the image classification value for the current spectral image has a value requiring purification processing as a result of comparison, determining the water quality of the stored water at the current time as requiring purification processing.

25. The method of claim 23, further comprising:
if the water quality is determined as requiring purification processing, creating control information for the purification processing; and
transmitting the created control information to a purification device for the purification processing.
